(19) United States
(12) Patent Application Publication
(10) Pub. No.: US 2024/0207362 A1
(43) Pub. Date: Jun. 27, 2024

(54) Extracranial Methods For Facilitating Cerebrospinal Fluid Drainage

(71) Applicants: Institute for Basic Science (IBS), Yuseonggu (KR); Korea Advanced Institute of Science & Technology, Yuseonggu (KR)

(72) Inventors: Jin-Hui Yoon, Yuseonggu (KR); Hokyung Jin, Yuseonggu (KR); Ji Hoon Ahn, Yuseonggu (KR); Seon Pyo Hong, Yuseonggu (KR); Myung Jin Yang, Yuseonggu (KR); Gou Young Koh, Yuseonggu (KR)

(73) Assignees: Institute for Basic Science (IBS), Yuseonggu (KR); Korea Advanced Institute of Science & Technology, Yuseonggu (KR)

(21) Appl. No.: 18/383,601

(22) Filed: Oct. 25, 2023

Related U.S. Application Data
(60) Provisional application No. 63/419,243, filed on Oct. 25, 2022.

(51) Int. Cl. A61K 38/18; A61K 45/06; A61K 48/00; A61P 43/00

(52) U.S. Cl. CPC A61K 38/1866; A61K 45/06; A61K 48/005; A61P 43/00

(57) ABSTRACT

Described herein is a method of preventing or treating a neurodegenerative disease including administering an agent to a person in which the agent causes increase in outflow of cerebrospinal fluid through nasopharyngeal lymphatic plexus (NPLP), or an agent that causes contraction and relaxation of the circular smooth muscles covering the deep cervical lymphatic vessels (dcLVs).

### TECHNICAL FIELD

The present application relates to a method of increasing or recovering rate of or level of outflow of cerebrospinal fluid (CSF) from the central nervous system to the systemic circulation. The present application also relates to a method of diagnosing and treating neurodegenerative disease by assessing the level of CSF outflow and increasing the outflow in the patient identified as suffering from a reduced outflow level of CSF.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The range of cerebrospinal fluid (CSF) volume is 140-200 ml in the adult human. CSF is produced 400-600 ml/day from the choroid plexus and circulates brain parenchyma and spinal cord (Ref. 1 and 2). Thus, the fluid turns over 3-5 times/day. The central nervous system (CNS) comprises the brain, spinal cord, optic nerves, and retina, which require high energy for their delicate and fine activities. In turn, the CNS produces waste products and antigenic macromolecules including synaptic and myelin debris and misfolded proteins such as amyloid and hyperphosphorylated tau. These CNS-derived waste products and macromolecules are dissolved in brain interstitial fluids and CSF, which should be adequately drained into the systemic circulation. Excessive accumulation of these waste products and macromolecules including amyloid and hyperphosphorylated tau causes neurodegenerative diseases such as Alzheimer's disease (Ref. 3).

Traditionally, arachnoid villi and cribriform plate are known to be drainage routes of CSF (Ref. 1 and 2). However, since two research groups have rediscovered meningeal lymphatic vessels (mLVs) at the dura mater of the dorsal side skull in mice (Ref. 4 and 5), so-called “dorsal mLVs (FIG. 1)”, they have been suggested as a new drainage route of CSF. They (4, 5) showed that the dorsal skull mLVs are phenotypically similar to peripheral lymphatic capillaries. They also showed that the dorsal mLVs transport macromolecules and cells along the superior sagittal and transverse sinuses (4, 5). However, Ma and Proulx, and their colleagues (Ref. 6) did not find substantial CSF reuptake and drainage through the dorsal mLVs using the controlled low-rate and -volume stereotactic CSF tracer injections. These researchers instead suggested that CSF drains along cranial nerves as they exit the skull (5). In comparison, our group uncovered the basolateral mLVs as one of the main routes of CSF drainage (FIG. 2), and compared morphologic and functional differences between the dorsal mLVs and the basolateral mLVs in adult and aged mice (FIG. 3-5) (6). Importantly, the basolateral mLVs acquired lymphedematous characteristics and delayed CSF drainage with aging (FIG. 5) (6). These findings could explain the pathologic mechanism of neurodegenerative diseases such as Alzheimer's disease, that is, overaccumulation of waste products and macromolecules in the brain due to reduced CSF drainage through the impaired basolateral mLV over aging. However, the basolateral mLVs are regarded as a drainage route of CSF that circulates the posterior area of the brain and spinal cord (FIG. 6).

Thus, whether the mLVs exist or not for covering CSF drainage from the anterior (except cribriform plate area) and the middle cranial fossa areas of the CNS and skull has been poorly understood (FIG. 6). If they exist, how they connect to the extracranial lymphatics are unknown. In 2009, Pan et al (Ref. 7) reported the lymphatic vessels (LVs) in the human nasal cavity and nasopharynx, but they did not describe the relationship between CSF drainage and those LVs. In 2017, Ma and Proulx, and their colleagues (Ref. 8) described that they could observe the bright signal of the intracranially injected tracer P40D680 in the nasal cavity, and the tracer emanating from a plexus of lymphatic vessels on the pharynx, which tracked towards the deep cervical lymph node (dcLN), in the Prox1-GFP mice. Based on this finding, they propose that CSF can be drained through the pharyngeal LVs. But the identity of the pharyngeal LVs and their CSF outflow were not adequately elucidated. In 2022, Jacob and Thomas, and their colleagues (Ref. 9) reported the extended anterior mLV network around the cavernous sinus, with exit routes through the foramina of emissary veins using a light-sheet fluorescence microscopy (LSFM) imaging of mouse whole-head preparations after OVA-A555 tracer injection into the subarachnoid space. They also performed real-time vessel-wall (VW) magnetic resonance imaging (VW-MRI) after systemic injection of gadobutrol in the patients with neurological pathologies. However, because the tracing OVA-A555 in the fixed tissues and organs reflects the distribution of phagocytic macrophages rather than the distribution of mLVs and VW-MRI provided low resolution for the lymphatics, this study lacked detailed histological and functional information on the mLVs that are supposed to transport intracranial CSF to the extracranial compartment.

US patent application publication number 20190269758 METHODS AND COMPOSITIONS FOR MODULATING LYMPHATIC VESSELS IN THE CENTRAL NERVOUS SYSTEM (Ref. 10) relates to manipulation of mLVs for treating neurodegenerative diseases. However, this patent application does not disclose or suggest a method of increasing or recovering rate or level of CSF outflow from the central nervous system to the systemic circulation by modulating or manipulating the nasopharyngeal lymphatic plexus (NPLP)-deep cervical lymphatic vessel (dcLV) pathway of CSF.

US patent application publication number 20210311076 COMPOSITIONS AND METHODS OF DIAGNOSIS AND TREATMENT FOR NEUROLOGICAL DISEASES (Ref. 11) relates to manipulation of mLVs for treating neurodegenerative diseases. However, this patent application does not disclose or suggest a method of increasing or recovering rate or level of outflow of cerebrospinal fluid (CSF) from the central nervous system to the systemic circulation by modulating or manipulating the nasopharyngeal lymphatic plexus (NPLP)-dcLV pathway of CSF.

### SUMMARY OF THE INVENTION

The following embodiments and aspects thereof are described and illustrated in conjunction with systems, compositions and methods which are meant to be exemplary and illustrative, not limiting in scope.

Facilitating cerebrospinal fluid (CSF) drainage is valuable in preventing and treating neurodegenerative diseases, including Alzheimer's disease. Here, this invention provides information on how extracranial approaches and methods can facilitate CSF drainage.

This invention encompasses extracranial methods for facilitating CSF drainage through the nasopharyngeal lymphatic plexus (NPLP), and deep cervical lymphatic vessel (dcLV), leading to preventing and treating neurodegenerative diseases.
1. Applicant newly discovered that nasopharyngeal lymphatic plexus (NPLP) is a hub for CSF drainage through the skull base including the cribriform plate.
2. Applicant newly discovered the connecting routes for CSF drainage from the intracranial cavity to the NPLP.
3. Applicant newly discovered the connecting routes for CSF drainage from the intracranial cavity to the submucosa of the hard palate.
4. Applicant newly discovered that CSF drainage through the nasopharyngeal lymphatic plexus (NPLP)/medial side deep cervical lymphatic vessel (M-dcLV) route is greater in amount and faster in speed than through the basolateral mLV/lateral side (L)-dcLV route, emphasizing the importance of NPLP.
5. Applicant newly discovered that the NPLP is impaired with aging and that exogenous agent can restore decreased lymphatic vessel area with decreased CSF drainage to increased lymphatic vessel area with increased CSF drainage level.
6. Applicant newly discovered that contraction and relaxation of the circular smooth muscles covering the dcLVs can be modulated or regulated by stimulation and inhibition of the smooth muscle cells, peripheral nerves, neurotransmitters, mechanical stimulators, and gentle massage.
7. Applicant newly discovered that CSF drainage through the NPLP can be modulated by exogenous agents via an extracranial approach. CSF drainage can be facilitated by extracranial manipulations, administration of agents, and related regulators.

In one aspect, the present invention is directed to a method of increasing outflow of cerebral spinal fluid (CSF) from central nervous system comprising repairing or enlarging nasopharyngeal lymphatic plexus (NPLP) comprising: determining a subject in need of increased CSF outflow; and administering an effective amount of NPLP flow agent to the subject in need, whereby the amount of the agent repairs or enlarges NPLP of the subject, thereby increasing CSF outflow from the central nervous system to systemic circulation in the subject. The determining the subject in need of increased CSF outflow may include determining the subject to have a neurodegenerative disease or condition, determining the subject to have a risk factor for the neurodegenerative disease or condition, or both. The disease or condition may be cognitive decline with aging, Alzheimer's disease, Parkinson's disease, Huntington's disease, or stroke. In particular, the agent may be a VEGFR3 agonist, such as VEGF-C or VEGF-D, an analog, variant, or fragment thereof, or a combination of any of these. The agent may be also a fibroblast growth factor 2 (FGF-2), insulin-like growth factor 1 (IGF-1), hepatocyte growth factor (HGF), endothelin-1 (ET-1), angiopoietin-1, Tie2 agonist, neuropilins, or prostaglandin E2. The agent may be a protein, or a genetic vector that carries a gene encoding an agent polypeptide. The gene may encode vascular growth factor-C, angiopoietin-1, or Tie2 agonist.

In one aspect, the agent may be administered selectively at or near NPLP-dcLV space. The NPLP-dcLV space may be located in nasopharyngeal mucosa. The agent may be administered selectively at or near hard palate submucosa-mandibular lymph node space. The hard palate submucosa-mandibular lymph node space may be located in hard palate mucosa. The agent may be administered to the subject intrathecally to CSF space or trans-nasally to nasopharynx or trans-orally to hard palate submucosa. In this regard, the central nervous system of the subject may include soluble molecules, and wherein increasing the CSF outflow reduces the quantity of soluble molecules in the brain. The central nervous system of the subject may include amyloid-beta plaques, and wherein increasing the CSF outflow reduces the quantity of amyloid-beta plaques in the brain.

In another aspect, the present invention is directed to a method of increasing outflow of cerebral spinal fluid (CSF) from central nervous system comprising increasing contracting-relaxing of dcLV comprising: determining a subject in need of increased CSF outflow; and administering an effective amount of dcLV flow agent to the subject, whereby the amount increases contracting-relaxing of dcLV of the subject, thereby increasing CSF flow from the central nervous system to systemic circulation in the subject. The determining the subject in need of increased CSF outflow may include determining the subject to have a neurodegenerative disease or condition, determining the subject to have a risk factor for the neurodegenerative disease or condition, or both. The disease or condition may be cognitive decline with aging, Alzheimer's disease, Parkinson's disease, Huntington's disease, or stroke. The agent may be a stimulator of circular smooth muscle covering the dcLVs. The agent may be G protein-coupled receptor agonist phenylephrine or nitric oxide donor. In one aspect, agent may be administered to the subject transcervically, percutaneously, topically to neck muscles or neck nodes or neck spaces, or wherein the agent is mechanical application to the side of the neck.

In yet another aspect, the present application is directed to a method of preventing or treating or ameliorating a neurodegenerative disease or condition in a subject comprising repairing or enlarging nasopharyngeal lymphatic plexus (NPLP) comprising: determining the subject in need of increased CSF outflow from the central nervous system; and administering an effective amount of NPLP flow agent to the subject in need, whereby the amount of the agent repairs or enlarges NPLP of the subject, thereby increasing CSF outflow from the central nervous system to systemic circulation in the subject. The disease or condition may be cognitive decline with aging, Alzheimer's disease, Parkinson's disease, Huntington's disease, or stroke. In particular, the agent may be a VEGFR3 agonist, such as VEGF-C or VEGF-D, an analog, variant, or fragment thereof, or a combination of any of these. The agent may be also a fibroblast growth factor 2 (FGF-2), insulin-like growth factor 1 (IGF-1), hepatocyte growth factor (HGF), endothelin-1 (ET-1), angiopoietin-1, Tie2 agonist, neuropilins, or prostaglandin E2. The agent may be a protein, or a genetic vector that carries a gene encoding an agent polypeptide. The gene may encode vascular growth factor-C, angiopoietin-1, or Tie2 agonist.

The agent may be administered selectively at or near NPLP-dcLV space, including wherein the NPLP-dcLV space is located in nasopharyngeal mucosa. The agent may be administered to the subject intrathecally to CSF space or trans-nasally to nasopharynx. The agent may be administered selectively at or near hard palate submucosa-mandibular lymph node space. The hard palate submucosa-mandibular lymph node space may be located in hard palate mucosa. The agent may be administered to the subject intrathecally to CSF space or trans-nasally to nasopharynx or trans-orally to hard palate submucosa. In this regard, the central nervous system of the subject may include soluble molecules, and wherein increasing the CSF outflow reduces the quantity of soluble molecules in the brain. The central nervous system of the subject may include amyloid-beta plaques, and wherein increasing the CSF outflow reduces the quantity of amyloid-beta plaques in the brain.

In another aspect, the present invention is directed to a method of preventing or treating or ameliorating a neurodegenerative disease or condition in a subject comprising repairing or enlarging nasopharyngeal lymphatic plexus (NPLP) comprising: determining the subject in need of increased CSF outflow from the central nervous system; and administering an effective amount of dcLV flow agent to the subject, whereby the amount increases contracting-relaxing of dcLV of the subject, thereby increasing CSF outflow from the central nervous system to systemic circulation in the subject. The disease or condition may be cognitive decline with aging, Alzheimer's disease, Parkinson's disease, Huntington's disease, or stroke. The agent may be a stimulator of circular smooth muscle covering the dcLVs. The agent may be a G protein-coupled receptor agonist phenylephrine or nitric oxide donor. The agent that stimulates circular smooth muscle covering the dcLVs may increase or decrease myosin phosphorylation by activating myosin light chain kinase or activating myosin light chain phosphatase. The agent may be administered to the subject transcervically, percutaneously, topically to neck muscles or neck nodes or neck spaces, or wherein the agent is mechanical application to the side of the neck.

In another aspect, the present invention is directed to a method for determining a change in CSF outflow content or rate in a subject comprising: obtaining a biological sample to obtain a specimen of nasopharyngeal mucosa or hard palate mucosa on at least two separate times, assaying for presence and amount of CSF or an assayable substance in the CSF so as to obtain a value for each time, comparing the value of amount of CSF or the assayable substance, and determining the change in the amount of the value of CSF or the assayable substance, wherein a change in value of CSF or the assayable substance over time indicates changed CSF outflow content or rate. The biological sample may be obtained by swab or biopsy. The biological sample may include the NPLP or hard palate. The assaying may be carried out by imaging of NPLP or hard palate, or protein analysis of obtained biological samples.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows meningeal lymphatic vessels at the dorsal side of the skull (Dorsal mLVs) in the mouse. Immunofluorescence staining (IFS) image showing LYVE-1+ dorsal mLVs along the VE-cadherin+ superior sagittal sinus and transverse sinus.

FIG. 2 shows meningeal lymphatic vessels at the basolateral side of the skull (basolateral mLVs) in the mouse. IFS image showing LYVE-1+ basolateral mLVs (arrows) along the VE-cadherin+ petrosquamous sinus and sigmoid sinus.

FIG. 3 shows schematic diagrams depicting morphologic and functional differences between the dorsal mLVs and the basolateral mLVs in adult and aged mice.

FIG. 4 shows schematic diagram showing that the basolateral mLVs are “hot route” for CSF drainage (posterior part of brain) and are regressed in aged mice.

FIGS. 5A-5B show that CSF drainage of the basolateral mLVs is largely reduced in aged mice. (A) Intracranial injection of Alexa 488 (A488)-conjugated LYVE-1 antibody through cisterna magna (CM) for visualization of the basolateral mLVs. At 12 hr later, QD705 was injected into intracranial cavity, and the injected QD705 was visualized in the basolateral mLV 15 min later by a fluorescence microscope. (B) Comparison of QD705 drainage (arrows) in the A488-LYVE-1+ basolateral mLVs (arrowheads) between 3 months-old adult and 27 months-old aged mice.

FIG. 6 shows schematic diagram depicting known and unknown CSF drainage routes to the cervical lymph nodes.

FIGS. 7A-7B show distribution of exogenously injected dextran into the intracranial cavity in the adult mouse. (A) Schematic diagrams depicting intracranial injection of TMR-Dextran (10 kDa) through cisterna magna of 10 weeks-old Prox1-GFP mice. At 60 min later, the head was sampled. (B) Image showing the distribution of the TMR-Dextran in the intracranial cavity and extracranial cavity. Note that strong Prox1+ in the hippocampus and LVs around the naso- and oro-pharynx and hard- and soft-palates.

FIGS. 8A-8C show lymphatic plexus at nasopharynx but not oropharynx contains the CSF-derived-TMR-Dextran in the Prox1-GFP adult mouse. (A) Image showing LV distribution in the head and neck. (B) Image showing lymphatic plexus in the nasopharynx and oropharynx. (C) Image showing lymphatic plexus at nasopharynx but not oropharynx contains the CSF-derived-TMR-Dextran (reddish yellow arrowheads). Green arrowhead indicates lymphatic valve.

FIGS. 9A-9B show vital imaging showing dynamic and active dextran outflow from the intracranial cavity to the NPLP. (A) Schematic diagrams depicting intracranial injection of TMR-Dextran (10 kDa) through cisterna magna of 10 weeks-old Prox1-GFP mice. At 30 min later, semi-vital imaging was performed. (B) Video showing the CSF-derived-TMR-Dextran (reddish yellow arrowheads) dynamically drains through the NPLP.

FIG. 10 shows that the NPLP abundantly and selectively contains the CSF-derived fluorescent-microbeads in the Prox1-GFP adult mouse. The fluorescent-microbeads were injected into intracranial cavity through cisterna magna, and the NPLP was sampled 6 hours later. Dotted-line boxes are magnified as right panels.

FIG. 11 shows that the dcLVs have periodic lymphatic valves (green arrowheads) and lymphangions, and contains the TMR-Dextran, which was injected into the intracranial cavity via cisterna magna 30 min before. Note that the L-dcLV is connected to the basolateral LVs through the jugular foramen, whereas the M-dcLV is connected to the LVs derived from the nasopharynx. The injected dextran was detected not only in the dcLN but also within M- and L-dcLV.

FIGS. 12A-12B show that butterfly shape of the NPLP resides in the submucosa of the nasopharynx. (A) Images showing dorsal, ventral, and lateral views of the NPLP. Green lines outline major Prox1+/VEGFR3+/LYVE1+ LVs consisting of the NPLP. A flattened and condensed posterior nasal lymphatic plexus is located in front of the NPLP. (B) Schematic diagram showing structure and views of nasopharynx.

FIG. 13 shows coronal cross-section view of the NPLP (white arrowhead) in the Prox1-GFP adult mouse.

FIG. 14 shows schematic architecture of the NPLP. Dorsal, lateral, ventral, and coronal section views of NPLP are drawn. The NPLP is divided into head, body, and tail portions. Green lines outline the major LVs and valves for the CSF outflow.

FIG. 15 shows schematic 3D architecture of the NPLP. Green lines and vessels indicate inflow and outflow of CSF, and major LVs consisting of the NPLP.

FIGS. 16A-16C show discovery that an LV which is initiated from the pituitary gland area runs along the cavernous sinus connecting to the NPLP of Prox1-GFP mouse. (A) Schematic diagram showing the imaging region (red lines). (B) Serial images showing an LV initiated from the pituitary gland area connected to the NPLP. (C) Magnified view of red-lined box of (B) showing the lymphatic end in the Prox1+ pituitary gland.

FIG. 17 shows photo showing 3D structure of the connecting LV from the pituitary gland area to the NPLP, shown in FIG. 16.

FIGS. 18A-18B show discovery that LV runs along the pterygopalatine artery (PPA) connecting to the NPLP and the LVs of the hard palate. (A) Schematic diagram showing the imaging region (green lines). (B) Serial images showing a LV runs along the pterygopalatine artery (PPA) connecting to the NPLP and the LVs of the hard palate submucosa.

FIG. 19 shows photo showing 3D structure of the LV running along the pterygopalatine artery (PPA) connecting to the NPLP and the LVs of the hard palate submucosa, shown in FIG. 18.

FIGS. 20A-20D show discovery that Prox1+ LVs are located below the olfactory epithelium connecting to both the posterior nasal lymphatic plexus and NPLP of the nasopharynx. (A) Schematic diagram showing the imaging region (green lines). (B) The LV in the olfactory mucosa abundantly and selectively contains the CSF-derived fluorescent-microbeads in the Prox1-GFP adult mouse. The fluorescent-microbeads were injected into intracranial cavity through cisterna magna, and the olfactory was sampled 6 hours later. (C) The LV in the olfactory mucosa abundantly and selectively contains the CSF-derived QD705 in the Prox1-GFP adult mouse. The QD705 was injected 60 min before the sampling. (D) Image showing two connecting LVs between olfactory mucosa and NPLP and the posterior nasal lymphatic plexus.

FIG. 21 shows summary diagram for the discovery of three lymphatic routes from the intracranial cavity to extracranial NPLP. NPLP is a hub for CSF drainage. (1) an LV which is initiated from the pituitary gland area runs along the cavernous sinus connecting to the NPLP; (2) an LV runs along the pterygopalatine artery (PPA) connecting to the NPLP; (3) LVs located below the olfactory epithelium connecting to both the posterior nasal lymphatic plexus and the NPLP of the nasopharynx.

FIGS. 22A-22B show LVs in the submucosa of hard palate serve as a drainage route of CSF, which is connected to mandibular LN. (A) Image showing lymphatic vessels in submucosa of hard palate were connected to mandibula LN. And the injected dextran was detected not only in mandibular LN but also within lymphatic vessels in hard palate. (B) Image showing lymphatic vessels at submucosa of hard palate contains the CSF derived-TMR-Dextran.

FIG. 23 shows the lymphatic plexus in submucosa of the hard palate of primate, Cynomolgus monkey. Right image panels show the existence of CSF derived-Fluosphere in lymphatic plexus of the hard palate. Red arrows indicate CSF derived-Fluosphere (0.1 μm).

FIGS. 24A-24B show four lymphatic branches connecting NPLP and dcLVs in the Prox1-GFP mouse. (A) Images showing 4 lymphatic branches connecting NPLP and dcLVs before and after removal of the soft palate. (B) Schematic diagram showing 4 lymphatic branches connecting NPLP and dcLVs.

FIGS. 25A-25B show that NPLP exists in the submucosa of the nasopharynx in the primate, Cynomolgus monkey. (A) Gross image of the sagittal sectioned head and neck of the Cynomolgus monkey. (B) Immunofluorescence image of LYVE1+ NPLP and collagen IV+ BVs in the submucosa of the nasopharynx. Green lined box is magnified in the lower panel. White arrowheads indicate lymphatic valves.

FIGS. 26A-26B show that CSF drains through the NPLP and hard palate in the primate (Cynomolgus monkey) in vivo. (A) MRI image of monkey depicting contrast enhanced CSF by Gadospin P injection through cisterna magna. The red circle indicates region of interest for measuring signal enhancement by CSF tracer. (B) Quantification of signal enhancement for 3 hours from different regions shows the NPLP and hard palate is CSF drainage route.

FIGS. 27A-27C show that medial dcLV drains more CSF than lateral dcLV in the Prox1-GFP adult mice. (A) Schematic diagram depicting ligation of L-dcLV or M-dcLV. (B) Light and fluorescence microscopic images are shown in each dcLV ligation. (C) Comparisons of the signal intensity in the dcLN. n=9 per each group, P values were calculated by Brown-Forsythe and Welch ANOVA test.

FIGS. 28A-28D shows that medial dcLV drains CSF faster than lateral dcLV in the Prox1-GFP adult mice. (A) Schematic diagram depicting intracranial injection of TMR-Dextran and analyses timing. (B) Images showing the Dextran in each dcLV. (C) Schematic diagram depicting the Dextran in each dcLV in (B). (D) Comparisons of the signal intensity in the dcLVs. P values were calculated by Brown-Forsythe and Welch ANOVA test.

FIGS. 29A-29D show that intracisternal infused AAV-mVEGF-C increases Prox1+ lymphatic vessel area in the nasopharynx of young Prox1-GFP mice. (A) Schematic diagram depicting intracisternal injection of AAV-mVEGF-C-mCherry and analysis timing. (B), (C) Representative image of the NPLP and comparison of quantitative parameters. (D) Representative image of CSF tracer drainage in dcLN and comparisons. P values were calculated by Mann-Whitney test.

FIG. 30 shows that aged mice have altered morphology and reduced lymphatic signatures in the NPLP. Images showing the NPLP between 10 weeks-old and 80-88 weeks old aged Prox1-GFP mice.

FIGS. 31A-31C show transcriptome of lymphatic endothelial cells (LECs) from the young and aged NPLP. (A) 5 clusters of LECs were preserved in the aged NPLP. (B-C) List shows differential expressed genes in the aged NPLP. Differentially expressed genes were involved in pro-apoptosis and inflammatory signaling in the aged NPLP.

FIGS. 32A-32B show that phosphorylated tau (ptau) increases in the aged NPLP and number of apoptotic lymphatic endothelial cell increases in the aged NPLP. P values were calculated by Mann-Whitney test.

FIGS. 33A-33E show the effect of AAV-mVEGF-C-mCherry infection of the NPLP in aged mice. (A) Schematic diagram depicting intracisternal injection of AAV-mVEGF-C-mCherry and analysis timing. (B), (C) Representative images and comparisons that intracranial administration of AAV-VEGF-C-mCherry enlarges lymphatic vessel area of the NPLP. (D) Increased lymphatic vessels after AAV-mVEGF-C-mCherry infection are routes for CSF tracer. (E) Representative image of CSF tracer drainage in dcLN and comparisons. Note that AAV-mVEGF-C-mCherry infection increased CSF drainage after three weeks in aged mice. P values were calculated by two-tailed Mann-Whitney test.

FIG. 34 shows that dcLVs have well-developed lymphatic valves (green arrowheads) and lymphangions, and are finely covered with circular smooth muscle cells in the Prox1-GFP mice.

FIG. 35 shows that β3-tubulin⁺ peripheral nerve fibers are abundantly distributed along the dcLVs.

FIGS. 36A-36B show the innervating peripheral nerve fibers are sympathetic nerve fibers. (A) Innervating peripheral nerves are β3-tubulin+ and tyrosine hydroxylase (TH)+. The TH is a sympathetic nerve fiber marker. Vesicular acetylcholine transporter (VAChT) is negative in innervating peripheral nerve fibers along the dcLVs. (B) VAChT antibody is validated pulmonary bronchiole nerve staining.

FIGS. 37A-37B show that structure of downstream lymphatic vessels of the NPLP were not altered with aging. P values were calculated by two-tailed Mann-Whitney test. n.s., p>0.05.

FIGS. 38A-38C show that dcLVs are contracted by the potassium chloride solution. (A) Schematic diagram of the experimental procedure. The dcLV in the 10 weeks-old Prox1-GFP male mice were exposed by dissecting the muscles, applied saline (0.9% NaCl solution), and applied 0.1 M or 1.0 M of potassium chloride (KCl) 5 min later, and washed with saline 20 min later. (B) M-dcLV of Prox1-GFP mouse is contracted by KCl (white arrowheads). (C) M-dcLV of Prox1-GFP mouse that was injected with QD705 into the intracranial cavity 2 hrs before the imaging is contracted by KCl (white arrowhead).

FIGS. 39A-39B show that G protein-coupled receptor agonist phenylephrine or nitric oxide donor (sodium nitroprusside) contracts or dilates dcLVs. (A) Image panel shows no contraction or dilation by PBS (phosphate buffered saline) treatment, but phenylephrine or nitric oxide donor contract or dilate dcLV. (B) Graphs indicates dosage dependent contraction of dcLV by phenylephrine or dilation by nitric oxide donor. The amount of CSF tracer was decreased by phenylephrine or increased by nitric oxide donor. P values were calculated by two-way repeated measured ANOVA test.

FIGS. 40A-40C show low dosage of phenylephrine (10 nM) enhance CSF drainage measured by drained amount of CSF tracer in deep cervical lymph node. In higher dosage (5 mM), as shown in FIGS. 36, the amount of CSF tracer was decreased. Nitric oxide donor, sodium nitroprusside, did enhance CSF drainage with 3 μM but did not enhance or decrease CSF drainage with 30 μM. P values were calculated by two-way ANOVA test followed by Dunnett's T3 multiple comparison post-hoc test.

FIG. 41 shows extracranial regulation of CSF drainage.

FIG. 42 shows that the NPLP is a hub of CSF drainage. Directions of CSF outflow are indicated by green arrows.

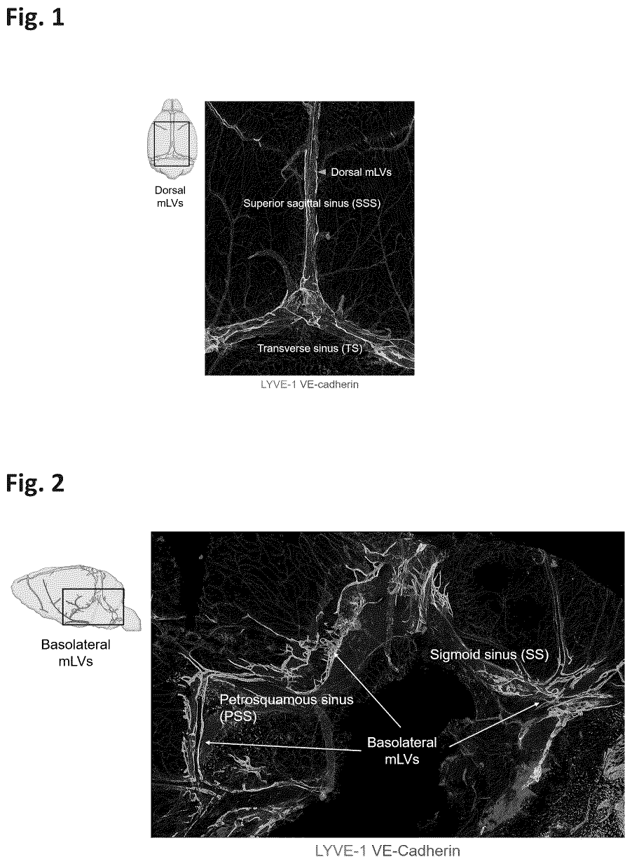

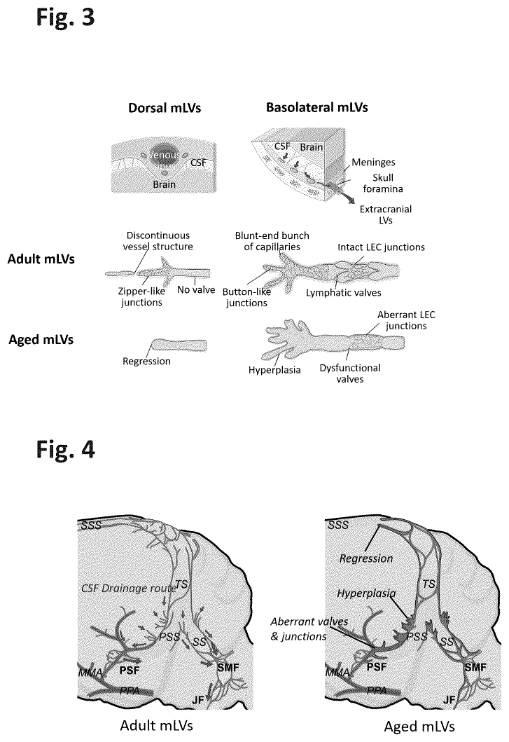

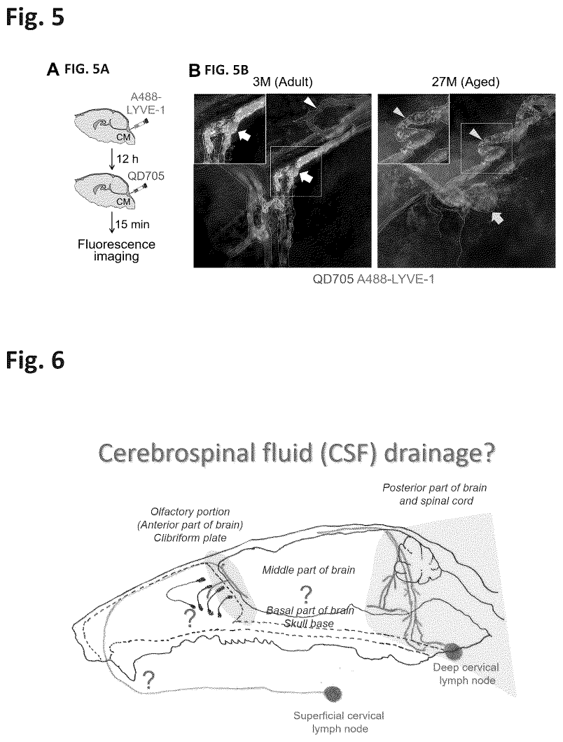

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N Y 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor NY, 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7; Paul W. Flint et al., Cummings Otolaryngology: Head and Heck Surgery, Elsevier Health Sciences, 2020 (ISBN 978-0323611794); Parviz Janfaza et al., Surgical Anatomy of the Head and Neck, Harvard University Press, 2011 (ISBN 978-0674058033).

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

“Treatment” or “treating” as used herein includes achieving a therapeutic benefit and/or a prophylactic benefit. By therapeutic benefit is meant eradication or amelioration of the underlying disorder or condition being treated. For example, in an individual with general age related deficiency in cognition or Alzheimer's Disease, therapeutic benefit includes partial or complete halting of the progression of the disorder or condition, or partial or complete reversal of the disorder or condition. Also, a therapeutic benefit is achieved with the eradication or amelioration of one or more of the physiological or psychological symptoms associated with the underlying condition such that an improvement is observed in the patient, notwithstanding the fact that the patient may still be affected by the condition. A prophylactic benefit of treatment includes prevention of a condition, retarding the progress of a condition (e.g., slowing the progression of Alzheimer's Disease or slowing the decline in cognitive abilities perhaps due to aging), or decreasing the likelihood of occurrence of a condition.

As used herein, the term “effective amount” can be an amount, which when administered, is sufficient to effect beneficial or desired results in the CNS, such as beneficial or desired clinical results, or enhanced cognition, memory, mood, or other desired CNS results. An effective amount is also an amount that produces a prophylactic effect, e.g., an amount that delays, reduces, or eliminates the appearance of a pathological or undesired condition. Such conditions include, but are not limited to, neurodegeneration. An effective amount can be administered in one or more administrations.

A “subject” or an “individual,” as used herein, is an animal, for example, a mammal. In some embodiments a “subject” or an “individual” is a human. In some embodiments, the subject suffers from Alzheimer's Disease or age-related cognitive disability.

In some embodiments, a pharmacological composition is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, trans-oral, parenteral, sublingual, topical, transmucosal or trans-nasal.

A “pharmaceutically acceptable carrier” or “pharmaceutically acceptable excipient” herein refers to any carrier that does not itself induce the production of antibodies harmful to the individual receiving the composition. Such carriers are well known to those of ordinary skill in the art. A thorough discussion of pharmaceutically acceptable carriers/excipients can be found in Remington's Pharmaceutical Sciences, Gennaro, A R, ed., 20th edition, 2000: Williams and Wilkins PA, USA. Exemplary pharmaceutically acceptable carriers can include salts, for example, mineral acid salts such as hydrochlorides, hydrobromides, phosphates, sulfates, and the like; and the salts of organic acids such as acetates, propionates, malonates, benzoates, and the like. For example, compositions of the invention may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate. “Pharmaceutically acceptable carrier” also includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Exemplary pharmaceutically acceptable carriers for injectable compositions can include salts, for example, mineral acid salts such as hydrochlorides, hydrobromides, phosphates, sulfates, and the like; and the salts of organic acids such as acetates, propionates, malonates, benzoates, and the like. Commonly used buffers include histidine, acetate, phosphate, or citrate. Under ordinary conditions of storage and use, these preparations can contain a preservative to prevent the growth of microorganisms. The prevention of the action of microorganisms can be brought about by various antibacterial and antifungal agents, for example, parabens, chlorobutanol; phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate, and gelatin.

For human administration, preparations meet sterility, pyrogenicity, general safety, and purity standards as required by FDA and other regulatory agency standards. The active compounds will generally be formulated for parenteral administration, e.g., formulated for injection via the intravenous, intramuscular, subcutaneous, intralesional, or intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

Upon formulation, solutions will be systemically or locally administered in a manner compatible with the dosage formulation and in such amount as is therapeutically effective based on the criteria described herein. The formulations are easily administered in a variety of dosage forms, such as the type of injectable solutions described above, but drug release capsules and the like can also be employed.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins, wherein the amino acid residues are linked by covalent peptide bonds.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides.

As used herein, “intracranial” refers to matter within the cranium or skull including the meninges and parenchyma as well as other structures.

As used herein, “extracranial” refers to matter that is not within the cranium, such as nasopharynx and medial deep cervical lymphatic vessel (dcLV).

As used herein, “exogenous agent” refers to a composition or methodology that can be applied to a subject so as to increase or stabilize flow of CSF. In particular, the exogenous agent affects flow in NPLP and/or dcLV. In this regard, “flow agent” is synonymous with an exogenous agent in particular as it increases or stabilizes outflow of CSF through NPLP and/or dcLV.

As used herein, “NPLP-dcLV” or “NPLP-dcLV path” refers to the extracranial area or space of lymph vessel or lymph structural system in which CSF outflow occurs from the NPLP to deep cervical lymph node occurring in the neck along the internal jugular vein with the carotid sheath.

NPLP and dcLV

As described herein, Applicant has discovered a new lymphatic system or route that functions in draining macromolecules, and debris from the central nervous system (CNS). In particular, reducing drainage through NPLP-dcLV vessels can reduce the outflow of cerebrospinal fluid (CSF), and can exacerbate symptoms of neurodegenerative diseases characterized by increases in concentration and/or accumulations of molecules in the central nervous system, for example, Alzheimer's disease (AD). Modulating lymphatic vessels to increase flow may alleviate cognitive impairment conditions due to old age, or symptoms of AD, including cognitive symptoms, accumulation of amyloid-beta plaques and hyperphosphorylated tau proteins.

Methods for treating, preventing, inhibiting, or ameliorating symptoms of neurodegenerative diseases or conditions associated with increased concentration and/or the accumulation of macromolecules, cells, and debris in the CNS are described. The methods may increase drainage by lymphatic vessel, and thus increase flow in CSF. The inventive methods are advantageous because they include one, several or all of the following benefits: (i) increased outflow and drainage of CSF; (ii) decreased accumulation of macromolecules, cells, or synaptic and myelin debris in the CNS (for example, decreased accumulation of amyloid-beta); and (iii) maintenance of or improvement in cognitive function (for example memory function) in a subject suffering from cognitive impairment condition due to old age, or is suspected of having, and/or at risk for dementia (such as in a neurodegenerative disease).

### CSF Flow and Agents for Increasing or Stabilizing Flow

As used herein, “flow” refers to a rate of perfusion through an area of the central nervous system of a subject. In some embodiments of the invention, “flow” can be measured as a rate at which a label or tracer in CSF perfuses through a particular area of the central nervous system. As such, flow can be compared between two subjects or two sets of conditions by ascertaining how quickly an injected label or tracer perfuses throughout a particular area or volume of the brain and/or other portion of the CNS. Additionally, “outflow” refers to the drainage of CSF into systemic circulation, and in particular through the inventive NPLP-cdLV pathway.

As used herein, “exogenous agent”, “agent” or “flow agent” broadly refers to classes of compositions that can increase the passage of substances into and out of lymphatic vessels, and thus can modulate flow in CSF. In particular, the invention is directed to an agent that specifically or generally increases flow of CSF in NPLP (“NPLP flow agent”) or dcLV (“dcLV flow agent”).

Without being limited by theory, it is contemplated, according to several embodiments herein, that removal of macromolecules through the NPLP-dcLV lymphatic vessels can keep their concentrations low in the CSF, allowing a gradient to clear macromolecules from the parenchyma. Furthermore, the higher the rate of fluid flow and drainage in the CNS, the higher the rate of clearance and/or the lower the concentration of cells, macromolecules, waste, and debris form the CNS.

Exogenous agents may increase the diameter of the NPLP-dcLV lymphatic vessels, and/or repair NPLP-dcLV lymphatic vessels, which increases or stabilizes rate or level of drainage, resulting in increased flow of the CSF. In some embodiments, exogenous agents enlarge or repair NPLP, and/or enhance contraction and relaxation of the dcLV, thus increasing net drainage, resulting in increased or stabilized flow of the CSF.

### NPLP Flow Agent

Examples of suitable NPLP flow agents for increasing CSF flow include, but are not limited to, vascular growth factor C (VEGF-C), vascular growth factor D (VEGF-D), fibroblast growth factor 2 (FGF-2), insulin-like growth factor 1 (IGF-1), hepatocyte growth factor (HGF), endothelin-1 (ET-1), angiopoietin-1, Tie2 agonist, neuropilins, prostaglandin E2, and further include viral vector-mediated gene transfer of VEGF-C or VEGF-D or FGF-2 or IGF-1 or HGF or ET-1 or angiopoietin-1.

VEGFR3, also known as FLT4, is a receptor tyrosine kinase, and its signaling pathway has been implicated in embryonic lymphatic development, and adult lymphangiogenesis. Upon binding of ligand, VEGFR3 dimerizes, and is activated through autophosphorylation. As such, VEGFR3 agonists are suitable for methods for treating, reducing the symptoms of, or preventing neurodegenerative diseases associated with accumulation of molecules in the brain, for example AD, in accordance with some embodiments herein. Accordingly, in some embodiments, such as methods or compositions for which increased drainage and flow are desired, a flow agent is a VEGFR3 agonist.

In particular, VEGF-C promotes the growth of lymphatic vessels (lymphangiogenesis). It acts on lymphatic endothelial cells (LECs) primarily via its receptor VEGFR-3 promoting survival, growth and migration. As such, VEGF-C is suitable as VEGFR3 agonist.

An effective amount of VEGFR3 agonist in accordance with the inventive methods can be understood in terms of its ability to enlarge or repair NPLP, so as to increase or stabilize flow of CSF, or to treat, ameliorate, or prevent various cognitive impairment conditions due to old age or neurodegenerative disease by increasing clearance of substances from the CNS. Accordingly, in the inventive methods, an effective amount of VEGFR3 agonist enlarges NPLP by at least about 2%, for example, at least about 2%, 3%, 4%, 5%, 6%, 7%, 8%, 9%, 10%, 15%, 20%, 25%, 30%, 35%, 40%, 45%, 50%, 55%, 60%, 65%, 70%, or 75%, including ranges between any two of the listed values. In some embodiments, an effective amount of VEGFR3 agonist increases flow of the CSF by at least about 2%, for example, at least about 2%, 3%, 4%, 5%, 6%, 7%, 8%, 9%, 10%, 15%, 20%, 25%, 30%, 35%, 40%, 45%, 50%, 55%, 60%, 65%, 70%, or 75%, including ranges between any two of the listed values.

In addition to VEGF-C, a VEGFR3 agonist may include VEGF-D. In one aspect, VEGF-C and VEGF-D together agonize VEGFR3, and can be provided in a single composition, or in separate compositions. In some embodiments, a VEGFR3 agonist includes an analog, variant, or functional fragment, such as a mutant, ortholog, fragment, or truncation of VEGF-C or VEGF-D.

The present application also discloses exogenous nucleotides encoding a VEGFR3 agonist, such as VEGF-C. In one aspect, a nucleotide encoding VEGF-C or VEGF-D as described herein is expressed in a subject in order to administer the VEGFR3 agonist to a subject. For example, an exogenous vector such as a retroviral, lentiviral, adeno-viral, or adeno-associated viral vector containing a nucleic acid encoding a VEGFR agonist as described herein can be inserted into a host nucleic acid of the subject. In some embodiments, the vector further comprises transcriptional machinery to facilitate the transcription of the nucleic acid encoding the VEGFR3 agonist.

In one aspect, the VEGFR3 agonist may include a modification, for example a glycosylation, PEGylation, or the like. In some embodiments, a composition for use in accordance with the methods described herein comprises the VEGFR3 agonist, and a pharmaceutically acceptable diluent or carrier.

The present invention is also directed to a method of applying a flow agent such as a chemical molecule or a biological molecule, and then assaying for the rate of flow of the CSF through the NPLP or the NPLP-dcLV pathway, wherein if the flow or outflow is greater than before the application of the flow agent, then the flow agent is deemed to be an effective agent that increases flow of CSF in the region.

Thus, in one aspect, the present invention is directed to a method of determining a CSF agent that increases outflow of CSF through the NPLP or the NPLP-dcLV pathway.

dcLV Flow Agent

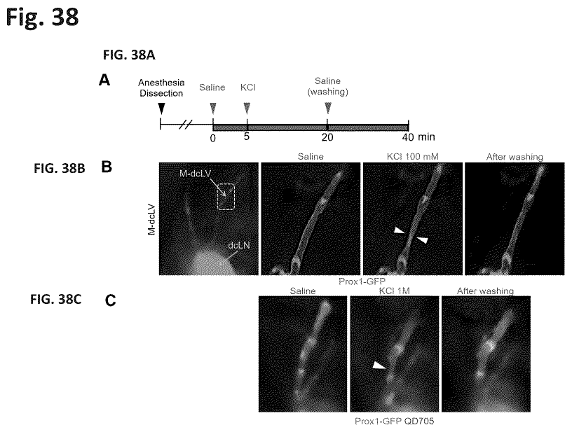

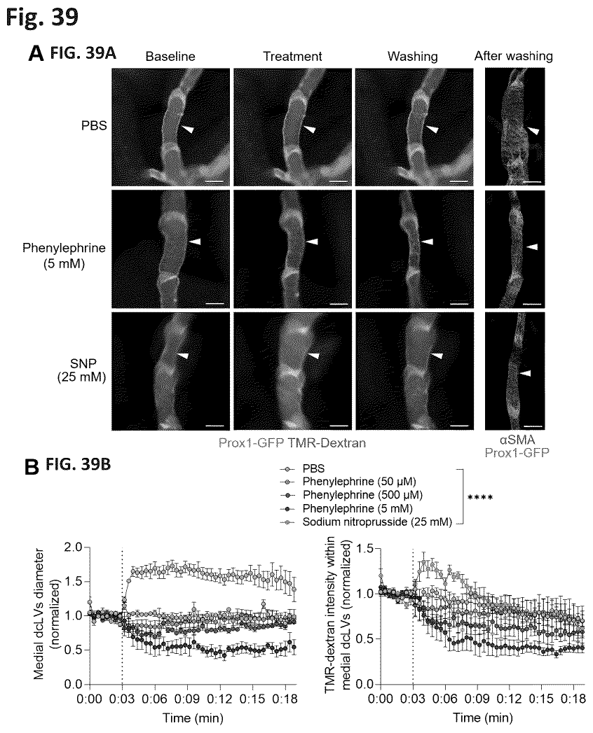

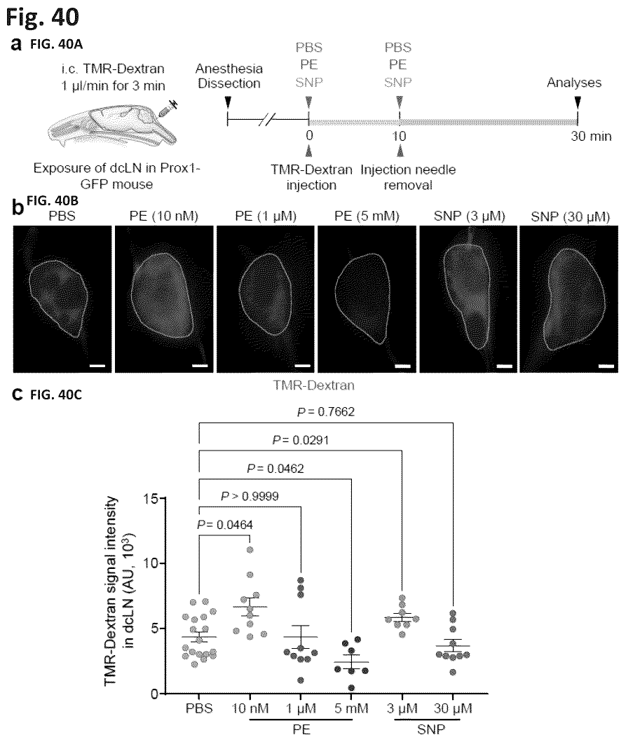

Some dcLV flow agents can be described as follows. dcLVs are strongly but transiently contracted by 0.1 and 1.0 M of potassium chloride (KCl) (FIG. 38). In addition, G protein-coupled receptor agonist phenylephrine or nitric oxide donor (sodium nitroprusside) contracts or dilates dcLVs and the amount of CSF tracer can be modulated (FIG. 39). Low dosage of phenylephrine (10 nM) increased the amount of drained CSF but reduced the amount of drained CSF with high dosage (5 mM) measured by accumulated CSF tracer in dcLN (FIG. 40). Low dosage of nitric oxide donor (3 μM), sodium nitroprusside, enhanced CSF drainage but no change is observed with 30 μM of nitric oxide donor (FIG. 40).

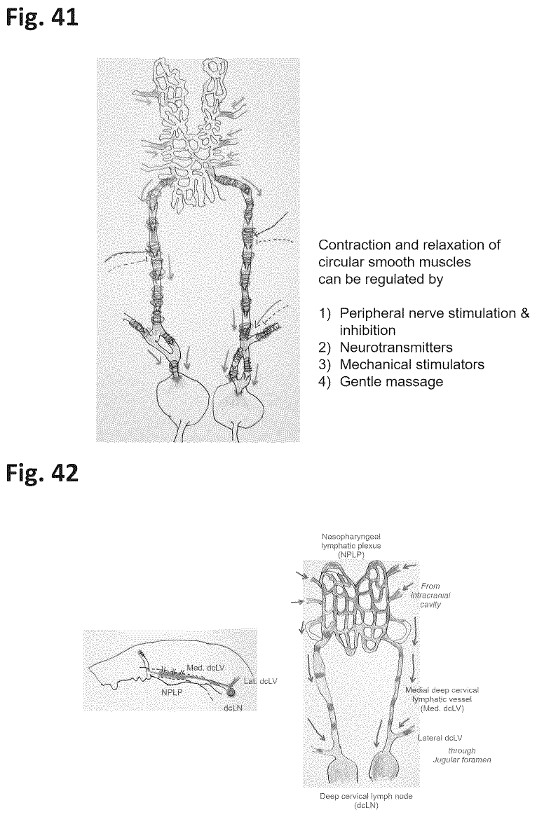

Contraction and relaxation of the circular smooth muscles covering the dclVs can be regulated by 1) stimulation and inhibition of the smooth muscle cells or peripheral nerves, 2) neurotransmitters, 3) mechanical stimulators, and 4) gentle massage of the neck area near cdLV. CSF drainage can be facilitated by extracranial manipulations, administration of agents, and regulators (FIG. 41). Considering that there is no prominent change in aged dcLVs, the reduced CSF drainage can be enhanced by extracranial manipulations of dcLVs.

The administered composition may include a single unit dose of flow agent effective for increasing or repairing flow, increasing clearance or reducing accumulated amyloid-beta plaques. In some embodiments, the effective amount of flow agent is about 0.00015 mg/kg to about 1.5 mg/kg, including any other amount or range contemplated as a therapeutically effective amount of a compound as disclosed herein. The range may be greater or less than the range of about 0.00015 mg/kg to about 1.5 mg/kg.

The mechanical stimulation may include a single or multiple vibrations for increasing or repairing flow, or increasing clearance or reducing accumulated amyloid-beta plaques or phosphorylated tau in CSF. In some embodiments, the effective acceleration of CSF through dcLV is about 0.001 m/s² to about 2.5 m/s², including any other range contemplated as a therapeutically effective acceleration as disclosed herein. The range may be greater or less than the range of about 0.001 m/s² to about 2.5 m/s². The mechanical stimulation is not limited to mechanical vibration, and may include massage or any other mechanical methods to allow dcLV to contract and release the dcLV. A vibration or massage may be performed so as to increase the flow of CSF through the dcLV. A vibration machine that is applied for human use is available in the art.

The present invention is also directed to a method of applying a flow agent such as a biological or chemical molecule or physical pressure methods, and then assaying for the rate of flow of the CSF through dcLV, wherein if the flow or outflow in dcLV region is greater than before the application of the flow agent, then the flow agent is deemed to be an effective agent that increases flow of CSF in the region. In one aspect, the present invention is directed to a method of determining a CSF agent that increases outflow of CSF through dcLV.

### Routes of Administration

The flow agents according to the invention may be administered to a subject using any of a number of suitable routes of administration, provided that the route of administration administers the flow agent to the NPLP-dcLV path of a subject.

By “administering to the NPLP-dcLV path of a subject,” as used herein, it is not necessarily required that a flow agent be administered directly to the NPLP-dcLV path space, but rather, this term encompasses administering a flow agent directly and/or indirectly to the NPLP-dcLV path space. It is contemplated that administering the flow agent so that it is in fluid communication with the NPLP-dcLV path space of the subject in accordance with some embodiments herein typically by administering the flow agent optionally in the brain, the flow agent will be administered to the NPLP-dcLV path space. Accordingly, in one aspect, the flow agent is not administered systemically. In another aspect, the flow agent is not administered systemically, but rather is administered to a fluid, tissue, or organ in fluid communication with the NPLP-dcLV path space. In some embodiments, the flow agent is not administered systemically, but rather is administered to the CNS. In some embodiments, the flow agent is administered to the CNS, but is not administered to any organ or tissue outside of the CNS. In some embodiments, the flow agent is not administered to the blood.

In some embodiments, the flow agent is administered nasally orally so as to more directly impact the NPLP. For example, the flow agent can be provided in a nasal spray, or can be contacted directly with a nasal mucous membrane.

In some embodiments, the flow agent is administered through contacting with CSF of the subject. For example, the flow agent can be directly injected into CSF of a patient (for example intrathecally). Suitable apparatuses for injection can include a syringe, or a pump that is inserted or implanted in the subject and in fluid communication with CSF. In another aspect, the flow agent, may be in a slow-release gel, is implanted in a subject so that it is in fluid communication with CSF of the subject, and thus contacts the CSF. Topical administration in particular to the neck region to affect the muscle surrounding dcLV is also contemplated. Nonlimiting examples for topical administration include creams, lotions, gels, salves, sprays, dispersions, suspensions, pastes and ointments.

In some aspects, the flow agent is administered transcranially. For example, the flow agent such as in a gel formulation can be placed on an outer portion of the subject's skull, and can pass through the subject's skull. In some embodiments, the flow agent is contacted with a thinned portion of the subject's skull to facilitate transcranial delivery.

In another aspect, the flow agent is administered by expressing a nucleic acid encoding the flow agent in the subject. A vector including a nucleic acid, for example a viral vector such as a retroviral vector, lentiviral vector, or adenoviral vector, or adeno-associated viral vector (AAV) can be administered to a subject as described herein, for example via injection or inhalation. In some embodiments, expression of the nucleic acid is induced in the subject, for example via a regulator of transcription.

In some embodiments, the flow agent is administered selectively to the NPLP-dcLV path space of the subject. As used herein, administered “selectively” indicates that the flow agent is administered preferentially to the indicated target compared to other tissues or organs. As such, direct injection to extracranial NPLP-dcLV path would represent “selective” administration, whereas administration to CSF in general via a spinal injection would not. In some embodiments, the flow agent is administered selectively to the NPLP-dcLV path space, and not to portions of the CNS outside of the NPLP-dcLV path space, nor to any tissues or organs outside of the CNS. In some embodiments, the flow agent is administered selectively to the CNS, and not to tissue or organs outside of the CNS such as the peripheral nervous system, muscles, the gastrointestinal system, or musculature.

In some embodiments, the flow agent is administered to area surrounding neck area of the NPLP-dcLV path of the subject. As used herein, surrounding neck area of the NPLP-dcLV indicates the tissues around lymphatic vessels from the NPLP to the deep cervical lymph nodes.

In some embodiments, the flow agent is administered to the surrounding neck area of the mandibular lymph node. As used herein, surrounding neck area of the mandibular lymph node indicates the tissues around afferent lymphatic vessels of the mandibular lymph nodes. For example, lymphatics from the hard palate to the mandibular lymph nodes are included.

For any of the routes of administration listed herein in accordance with the method of the present invention, it is contemplated that a flow agent can be administered in a single administration, or in two or more administrations, which can be separated by a period of time. For example, in some embodiments, the flow agent as described herein can be administered via a route of administration as described herein hourly, daily, every other day, every three days, every four days, every five days, every six days, weekly, biweekly, monthly, bimonthly, and the like. In some embodiments, the flow administration is administered in a single administration, but not in any additional administrations.

Some embodiments include methods of making a composition or medicament including a flow agent as described herein suitable for administration according to a route of administration as described herein. For example, in some embodiments, VEGFR3 agonist is prepared for nasal administration, administration to the CSF, or transcranial administration.

Particularly preferred methods of administration of the agents include without limitation intrathecal approach to CSF space, transnasal or transoral approach to nasopharynx, transcervical or percutaneous or topical approach to neck muscles or neck nodes or neck spaces.

### Neurodegenerative Diseases and Conditions

The present invention is useful for treating, preventing, inhibiting, ameliorating, or reducing the symptoms of one or more neurodegenerative diseases. These diseases can occur in subjects, for example humans, as well as non-human animals, such as non-human mammals, and non-human primates in particular.

In some embodiments, neurodegenerative diseases associated with accumulation of macromolecules, cells, and debris in the CNS are treated, prevented, inhibited, or reduced by methods that increase flow, drainage, and/or clearance in NPLP-dcLV lymphatic vessels. In some embodiments, neurodegenerative diseases associated with accumulation of macromolecules, cells, and debris in the CNS are treated, prevented, inhibited, or reduced. Examples of neurodegenerative diseases include cognitive decline with aging, Alzheimer's disease, Parkinson's disease, Huntington's disease, or stroke.

In some embodiments, the neurodegenerative disease can be prevented, treated, or ameliorated prophylactically. Accordingly, a subject having one or more risk factors for the neurodegenerative disease can be determined to be in need of receiving the method of treatment described herein. For example, a subject may have accumulated amyloid-beta plaques in their CNS, and may benefit from increased flow, increased drainage, increased clearance and/or reduction of amyloid-beta plaques, even if they do not yet have an AD diagnosis based on cognitive symptoms. A number of risk factors for AD are suitable as risk factors in accordance with methods, compositions, and uses of some embodiments herein, for example familial AD, a genetic marker for AD, or a symptom of AD such as early dementia. The foremost risk factor for sporadic AD is age. However, increased risk of this form of AD has also been attributed to diverse genetic abnormalities.

### Methods for Increasing Flow

The inventive method can include determining whether the subject is in need of increased fluid flow in the central nervous system. Further, the inventive method can include determining whether the subject is in need of increased drainage of CFS. If the subject is in need of increased fluid flow or drainage, the methods can include administering an effective amount of VEGFR3 agonists or mechanical stimulations to a NPLP-dcLV path space of the subject. Thus, fluid flow in the central nervous system of the subject can be increased as well as level or rate of CFS outflow. In some embodiments, the VEGFR3 agonist comprises VEGF-C or VEGF-D or an analog, variant, or fragment thereof. It is also contemplated that for in some embodiments herein, FGF2 can be substituted for the indicated VEGFR3 agonist in order to increase flow, or can be used in addition to a VEGFR3 agonist in order to increase flow. In some embodiments, mechanical stimulation can be applied to neck area around the NPLP-dcLV path.

A subject can be determined to be in need of increased fluid flow or outflow by determining whether the subject has cognitive impairment related to old age or not, a neurodegenerative disease, or is at risk of developing a neurodegenerative disease. The disease can be associated with the increased concentrations and/or accumulation of molecules or cells or debris in the CNS, for example Alzheimer's Disease (AD). In some embodiments, the subject can be determined to be at risk for the disease, for example through having familial occurrence of the disease, by having one or more genetic markers associated with the disease, through advanced age, or by exhibiting symptoms of the disease, for example early dementia in the case of AD.

As used herein, “advanced age” refers to an age characterized by a decrease in memory function, decrease in CSF production, substantial increases in neuronal senescence, and in the context of some embodiments, can include at least 65 years of age in a human, for example, at least 60, 65, 70, 75, 80, or 85, including ranges between any of these values. In some embodiments, determining whether the subject is in need of increased fluid flow or outflow comprises determining the subject to have a neurodegenerative disease such as AD. In some embodiments, determining whether the subject is in need of increased fluid flow or outflow comprises determining the subject to have a risk factor for the neurodegenerative disease associated with the increased concentration and/or accumulation of molecules or macromolecules or cells or debris in the CNS as described herein. In some embodiments, determining whether the subject is in need of increased fluid flow or outflow comprises determining the subject to have a risk factor, and also determining the subject to have the disease itself.

In some embodiments, the neurodegenerative disease or condition is selected from the group consisting of at least one of the following: cognitive impairment possibly due to old age, Alzheimer's disease (AD), dementia, Parkinson's disease, Huntington's disease, or stroke. In some embodiments, the neurodegenerative disease is Alzheimer's disease. In some embodiments, the risk factor is a risk factor for Alzheimer's disease as described herein. In some embodiments, the VEGFR3 agonist and/or FGF2 is administered to the subject after determining that the subject has a risk factor for the neurodegenerative disease (even if the subject does not necessarily have the disease itself), for example for prophylactic treatment or prevention. In some embodiments, the VEGFR3 agonist and/or FGF2 is administered to the subject after determining that the subject has the neurodegenerative disease.

Without being limited by theory, it is contemplated, according to several embodiments herein, that systemic administration is not required for the VEGFR3 agonist and/or FGF2 to effectively modulate NPLP-dcLV lymphatic vessel size and drainage, or flow. Accordingly, in some embodiments, the VEGFR3 agonist and/or FGF2 is administered selectively to the NPLP-dcLV path space of the subject. In some embodiments, the VEGFR3 agonist and/or FGF2 is administered to the space, inside or outside the CNS. In other embodiments, the VEGFR3 agonist and/or FGF2 is administered to the NPLP-dcLV path space, but is not administered to the blood. In some embodiments, the VEGFR3 agonist and/or FGF2 is administered to the subject by a route selected from the group consisting of at least one of the following: topical, intrathecal, nasal administration, transoral, transcranial administration, contact with cerebral spinal fluid (CSF) of the subject, pumping into CSF of the subject, implantation into the skull or brain, contacting a thinned skull or skull portion of the subject with the VEGFR3 agonist and/or FGF2, or expression in the subject of a nucleic acid encoding the VEGFR3 agonist and/or FGF2, or a combination of any of the listed routes. In some embodiments, it is the VEGFR3 agonist that is administered. In some embodiments, the VEGFR3 agonist is selected from the group consisting of at least one of the following: VEGF-C, VEGF-D, or an analog, variant, or functional fragment thereof.

In some embodiments, the administration of the VEGFR3 agonist results in an increase in NPLP-dcLV lymphatic vessel diameter, NPLP-dcLV path lymphatic vessel number, NPLP-dcLV path lymphatic vessel drainage, or amelioration of symptoms of a neurodegenerative disease or condition. For example, in some embodiments, the administration of the VEGFR3 agonist increases diameter of the NPLP lymphatic vessel is increased by at least about 5%, for example at least about 5%, 10%, 15%, 20%, 25%, 30%, 35%, 40%, 45% or 50%, including ranges between any two of the listed values. In some embodiments, an average diameter of a population of NPLP-dcLV lymphatic vessels of the subject is increased by a value noted herein. In some embodiments, the administration of the VEGFR3 agonist increases CSF flow in the central nervous system, including CSF outflow in the subject, comprising increasing a rate of perfusion of fluid throughout an area of the subject's brain.

In some embodiments, the mechanical stimulations to surrounding neck area of the NPLP-dcLV path results in an increase of CSF flow, or amelioration of symptoms of a neurodegenerative disease or condition. For example, in some embodiments, the administration of the mechanical stimuli increases CSF flow in the central nervous system, including CSF outflow in the subject, by at least about 5%, for example at least about 5%, 10%, 15%, 20%, 25%, 30%, 35%, 40%, 45% or 50%, including ranges between any two of the listed values. In some embodiments, an average CSF flow rate of NPLP-dcLV lymphatic vessels of the subject is increased by a value noted herein. Here, mechanical stimulations include vibration, ultrasound stimulation, light stimulation, electric stimulation, magnetic stimulation, temperature stimulation and are not limited to the stimulation listed herein.

Increasing clearance can reduce macromolecules such as amyloid beta plaques, or decrease the rate of their accumulation. Without being limited by theory, it is contemplated that by clearing soluble amyloid beta from the CNS, a gradient will favor solubilization of amyloid beta plaques, so that fluids in the CNS continue to flow and the CNS continues to be cleared, amyloid beta plaques can diminish, or the rate of increase can be reduced. Thus, decreases of amyloid-beta plaques can represent a decrease in an etiology of a disease caused by amyloid-beta plaques.

Through increased fluid flow, the quantity of accumulated amyloid-beta plaques in the subject can be reduced, or the rate of accumulation can be reduced. In some embodiments, the quantity of accumulated amyloid-beta plaques, or the rate of accumulation, is reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values.

### Detecting Alteration in CSF Flow

The NPLP is a newly discovered hub for CSF drainage through the skull base including cribriform plate. The hard palate submucosa is a newly discovered route for CSF drainage to the mandibular lymph node. Therefore, it is within purview of the present invention to assay for a change in CSF flow rate in a person at these loci.

In one aspect, a swab or biopsy made be made of the nasopharyngeal mucosa or hard palate submucosa area so that a specimen of cells may be obtained, which should include NPLP or hard palate submucosa lymphatic cells. The CSF content of the sample is assayed or measured separately in time, and compared from time to time as the assay is repeated in a person and the results are compared.

As it is discovered that CSF outflow has lessened over a time, then an exogenous agent may be administered to increase CSF outflow in the subject.

The assay method may include imaging the NPLP with a tracer molecule and viewing through a microscope. Other methods may include without limitation, proteomic analysis of CSF specific proteins. Levels of selected proteins in the CSF can be measured from time to time to determine their amounts, wherein as the amounts of selected proteins are decreased or increased, indicates the status of level and quality of drainage of CSF. An example of a selected protein to monitor may include B2 transferrin in the obtained CSF sample. In particular, and for example, increased amount of B2 transferrin present in the sample over time indicates improved CSF drainage function. Decreased amount of B2 transferrin present in the sample over time indicates impaired CSF drainage function. Other proteins can be included without limitation.

### Facilitating Cerebrospinal Fluid (CSF) Drainage

Facilitating cerebrospinal fluid (CSF) drainage is valuable in preventing and treating neurodegenerative diseases, including Alzheimer' disease. Here, this invention provides information on how extracranial approaches and methods can facilitate CSF drainage. This invention encompasses the extracranial methods facilitating CSF drainage through the nasopharyngeal lymphatic plexus (NPLP) and the deep cervical lymphatic vessel (dcLV) for preventing and treating neurodegenerative diseases.

In one aspect, the invention is directed to a method of preventing or treating a central nervous system disease by improving CSF drainage in a person by repairing or at times enlarging NPLP. The CNS disease may be Alzheimer's disease, Parkinson's disease, or Huntington's disease. The NPLP may be recovered by administering to a person in need thereof an agent such as without limitation vascular growth factor C (VEGF-C), vascular growth factor D (VEGF-D), fibroblast growth factor 2 (FGF-2), insulin-like growth factor 1 (IGF-1), hepatocyte growth factor (HGF), endothelin-1 (ET-1), angiopoietin-1, Tie2 agonist, neuropilins, prostaglandin E2, viral vector-mediated gene transfer of VEGF-C or VEGF-D or FGF-2 or IGF-1 or HGF or ET-1 or angiopoietin-1.

The contraction and relaxation of the circular smooth muscles covering the dcLVs may be regulated by stimulation and inhibition of smooth muscle cells, such agent being without limitation an agent increasing or decreasing myosin phosphorylation by activating myosin light chain kinase or activating myosin light chain phosphatase by activating voltage-operated Ca2+ channels (KCl) or G protein-coupled receptor agonist (phenylephrine) or stimulating cGMP-dependent protein kinase (nitric oxide or acetylcholine).

The contraction and relaxation of the circular smooth muscles covering the dcLVs may be regulated further by stimulation and inhibition of peripheral nerves, such agent being able to interfere with depolarization or exocytosis of synaptic vesicles such as without limitation botulinum toxin or tetanus toxin.

The contraction and relaxation of the circular smooth muscles covering the dcLVs may be regulated also by stimulation and inhibition of neurotransmitter, such agent being without limitation agonist, antagonist of neurotransmitter receptor or agonist, antagonist of degradation enzymes at the circular smooth muscle covering the dcLV such as agonist or antagonist of norepinephrine.

The contraction and relaxation of the circular smooth muscles covering the dcLVs may be regulated also by mechanical stimulators such as without limitation vibrators, high- or low-frequency electrical stimulators, optogenetic stimulations by specific wavelength lights.

Delivery method of the agents may be without limitation intrathecal approach to CSF space, transnasal or transoral approach to nasopharynx, transcervical or percutaneous or topical approach to neck muscles or neck nodes or neck spaces.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Example 1: Facilitating cerebrospinal fluid (CSF) drainage is valuable in preventing and treating neurodegenerative diseases, including Alzheimer' disease. Here, this invention provides information on how extracranial approaches and methods can facilitate CSF drainage.

This invention encompasses the extracranial methods facilitating CSF drainage through the nasopharyngeal lymphatic plexus (NPLP) and the deep cervical lymphatic vessel (dcLV) for preventing and treating neurodegenerative diseases.

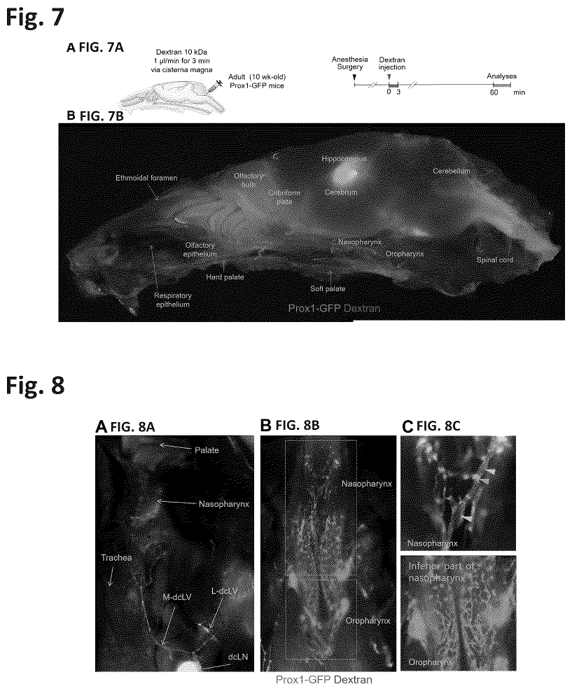

An anesthetized mouse (10 weeks-old male Prox-1 GFP mouse) was laid prone on a stereotaxic frame under a microscope equipped with a heating pad. The head was adjusted to a 90° angle to the body axis with the help of a mouthpiece to facilitate access to the cisterna magna. After a skin incision to the midline of the posterior neck, muscle layers were carefully separated with microreactors. The atlanto-occipital membrane overlying the cisterna magna was superficially penetrated using a 33-gauge NanoFil needle (World Precision Instruments) and then 3 μl of tetramethlyrhodamine (TMR)-conjugated 10 kDa MW dextran (Invitrogen, D1816) was infused into the subarachnoid space at 1 μl/min for 3 min using a micro-syringe (88000, Hamilton) and a micro-infusion machine (Fusion 100, Chemyx Inc) (FIG. 7A). The needle was slowly removed after the mouse was left in position for 5 min to prevent CSF leakage. The muscle layers and neck skin were then sutured with 6-0 black silk (Ailee, SK617). At 60 min after the infusion, the head portion was dissected without cardiac perfusion or soaking in saline. After decapitating, the head was sagittally cut in half, and the fluorescence image of the sectioned head was acquired using a fluorescence stereo zoom microscope (AxioZoom V16, Carl Zeiss) (FIG. 7A).

As a result, the dextran was highly accumulated in the olfactory bulb, cribriform plate, dorsolateral side of the skull, upper spinal cord area, and skull base adjacent to the nasopharynx (FIG. 7B). Of note, a notable Prox1-GFP signal was detected nasopharynx, oropharynx, hard palate, and soft palate (FIG. 7B), suggesting that there abundant LVs in these regions.

Example 2: The same procedure in Example 1 was applied to an anesthetized mouse (10 weeks-old male Prox1-GFP mouse), and then the mouse was sacrificed by cutting the abdominal aorta at 60 min after the injection. After dissection of surrounding muscles with a surgical microscope (SZX16, Olympus), the LVs and the dextran in the nasopharynx and dcLN were imaged using the fluorescence stereo zoom microscope (FIG. 8). We found that medial and lateral side afferent LVs to dcLN exist, and named them medial deep cervical LV (M-dcLV) and lateral-dcLV (L-dcLV) (FIG. 8A). While L-dcLV was connected to the basolateral LVs through the jugular foramen, M-dcLV was connected to the LVs derived from the nasopharynx (FIG. 8A). The injected dextran was detected not only in dcLN but also within M- and L-dcLV (FIG. 8A). When we examined the nasopharynx in detail, the dextran was highly detected in the upper portion of nasopharynx but not in the lower portion of the nasopharynx and oropharynx areas (FIGS. 8B and 8C). Importantly, the well-arranged Prox1+ lymphatic plexus surrounding the nasopharynx was found (FIGS. 8B and 8C), and named it “nasopharyngeal lymphatic plexus (NPLP)”. NPLP had valves (FIG. 8C, green arrowhead), and the injected dextran was highly detected within NPLP at upper portion of the nasopharynx (FIG. 8C, reddish-yellow arrowheads). NPLP was connected to the oropharyngeal lymphatic plexus (OPLP), but no dextran signal was detected within OPLP (FIG. 8C). These findings indicated that NPLP but not OPLP serves as a drainage route of CSF, which is connected to dcLN through M-dcLV.

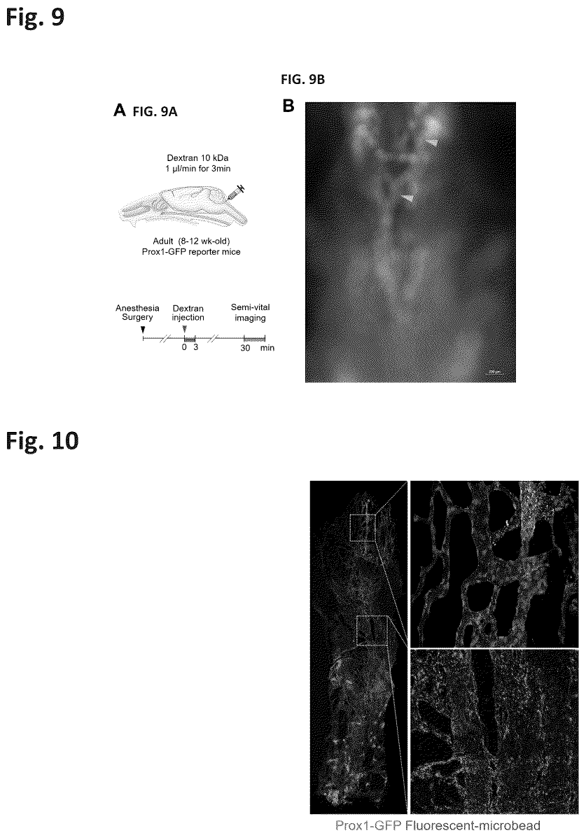

Example 3: The same procedures in Examples 1 and 2 were applied to an anesthetized mouse (10 weeks-old male Prox1-GFP mouse), and then the time-lapse vital imaging at the NPLP was obtained at 30 min after the injection using the fluorescence stereo zoom microscope (FIG. 9A). Flow of the injected dextran was detected within NPLP at the upper portion of the nasopharynx (FIG. 9B, reddish-yellow arrowheads).

Example 4: To verify the above findings, 3 μl of the fluorescent-microbeads (0.5 μm in diameter, F8887, Thermo-Fisher) containing solution was infused into the intracranial cavity at 1 μl/min for 3 min using a micro-syringe (88000, Hamilton) and infusion machine (Fusion 100, Chemyx Inc). Six hours later, the NPLP was sampled and found that the fluorescent-microbeads are abundantly and selectively with the NPLP (FIG. 10), confirming that the NPLP could serve as a hub for CSF drainage.

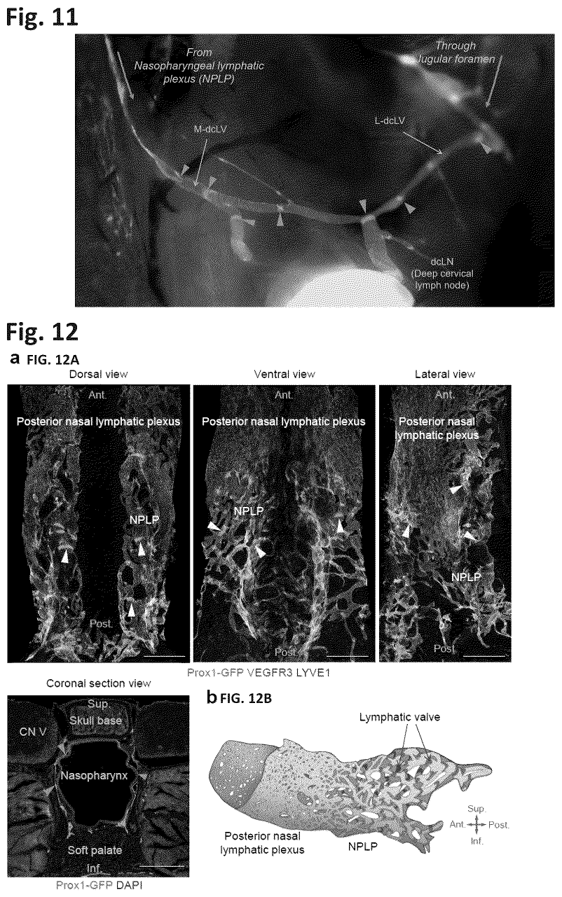

Example 5: The procedures for Example 1 were applied to an anesthetized mouse (10 weeks-old male Prox1-GFP mouse), and then the mouse was sacrificed by cutting the abdominal aorta at 60 min after the injection. After dissection of surrounding muscles with a surgical microscope (SZX16, Olympus), the LVs and the dextran in the proximal area of dcLN were imaged using a fluorescence stereo zoom microscope (FIG. 11). We found that medial and lateral side afferent LVs to dcLN, M-dcLV and L-dcLV (FIG. 11). While L-dcLV was connected to the basolateral LVs through the jugular foramen, M-dcLV was connected to the LVs derived from the nasopharynx (FIG. 11). The injected dextran was detected not only in dcLN but also within M- and L-dcLV (FIG. 11). Prox1+ dense lymphatic valves were periodically distributed within dcLVs (FIG. 11, green arrowheads).

Example 6: Dorsal, ventral and lateral views of NPLP and posterior nasal lymphatic plexus in the front part of nasopharynx were shown in FIG. 12. To obtain these images, immunofluorescence staining (IFS) of mouse NPLP in the 10 weeks-old male Prox1-GFP mice was performed. After anesthesia and right atrium puncture, ice-cold phosphate buffer saline (PBS) was perfused to the left ventricle to remove blood. After the PBS perfusion, 4% paraformaldehyde solution was injected through the left ventricle to fix the tissue. With a help of a surgical microscope (Stemi508, Zeiss), the nasopharyngeal mucosa was isolated by removing the surrounding skull, nerves, and soft tissues using fine forceps and surgical micro-scissor. Dorsal and ventral parts of the mucosa were cut in half with a transverse direction, and lateral part of the mucosa was cut in half with a sagittal direction. The collected tissues were fixed by 2% paraformaldehyde solution for 2 hours in 4 Celsius degree. After the fixation, the nasopharyngeal mucosa was incubated in 5% normal donkey serum (017-000-121, Jackson ImmunoResearch) for 1 hour at room temperature. To label lymphatic vessel markers, the nasopharyngeal mucosa was immersed in 5% normal donkey serum in which VEGFR3 antibody (AF743, R&D) and LYVE1 antibody (11-034, Angiobio) were dissolved at 1:400 at 4 Celsius degree for 12 hours. After PBS washing, the tissue block was incubated to normal donkey serum containing Alexa-594 conjugated anti-goat IgG antibody and Alexa-647 conjugated anti-rabbit IgG antibody (Jackson ImmunoResearch) for 12 hours in 4 Celsius degrees. After PBS washing, the tissues were covered with a mounting medium (H1200, Vector). Image was acquired by confocal microscope (LSM 800, Zeiss) with Plan-Apochromat 10×/NA 0.45 lens.

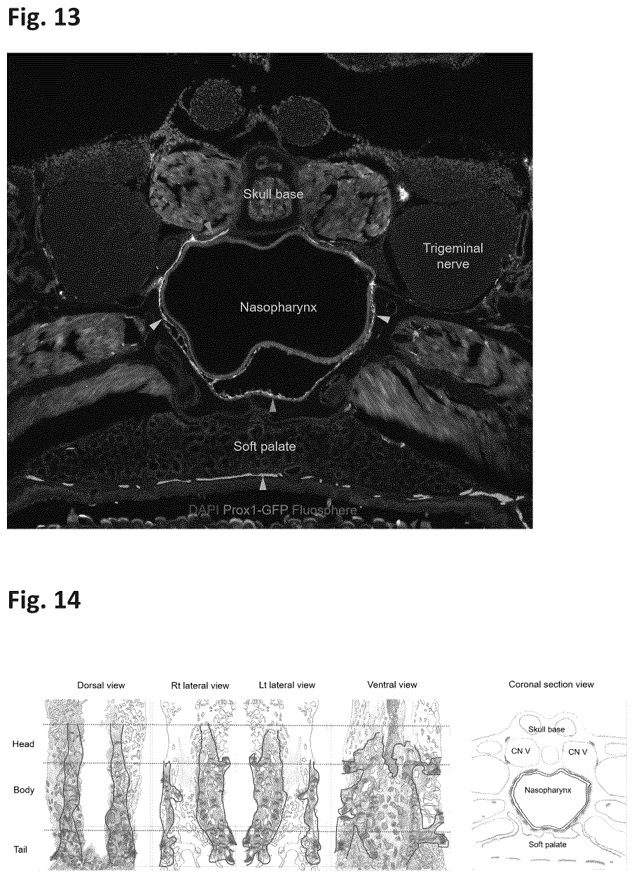

Cross section view of NPLP was shown in FIG. 13, (white arrowheads). The method for FIG. 12 was applied except for tissue sampling and section. Head was collected, surrounding skin was peeled off after the perfusion, and was submerged into 2% PFA solution post-fixation for 12 hours at 4 Celsius degree. For decalcification, the head was immersed in 0.5M EDTA solution for 48 hours at 4 Celsius degree. After decalcification, the head sample was dehydrated by submerging it in 30% sucrose solution for 48 hours. The head sample was embedded and frozen in a frozen section medium (Leica), and cut into a 30 μm section using a Cryocut Microtome (Leica). IFS was performed. The lymphatic vessels from the anterior portion to the nasopharynx constitute a posterior nasal lymphatic plexus. The NPLP has abundant lymphatic valves in the middle part of nasopharynx, and they are connected to the lymphatic vessels at the posterior part of the nasopharynx.

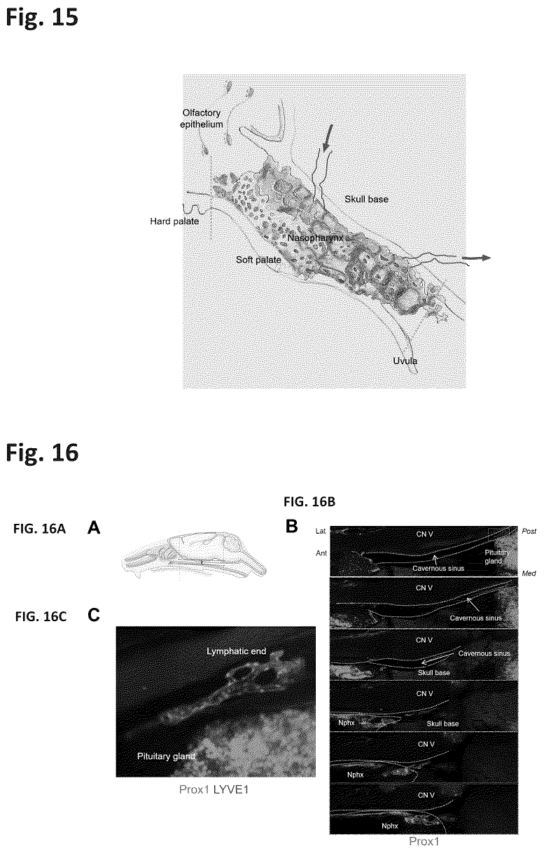

Based on FIG. 12 and FIG. 13, 2D and 3D schematic diagrams of NPLP are shown in FIG. 14 and FIG. 15.

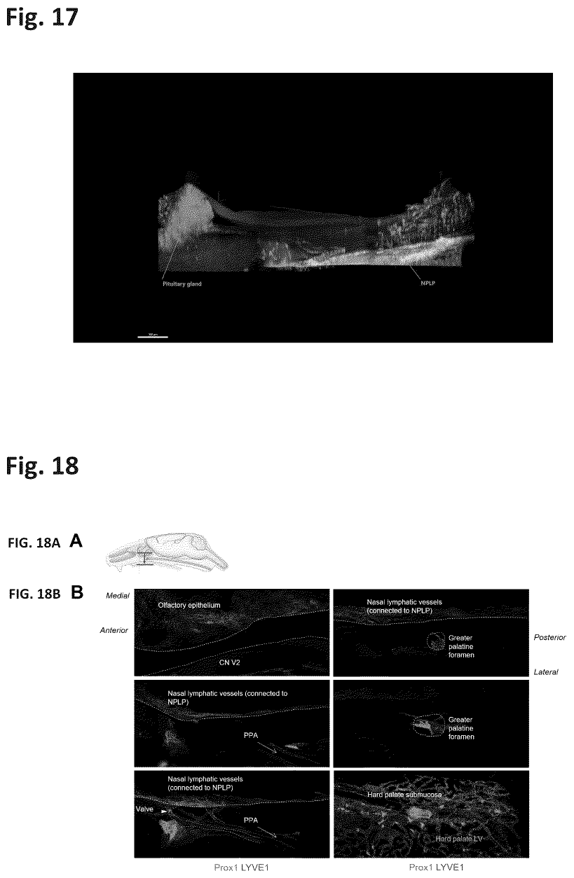

Example 7: Lymphatic vessels initiated from the pituitary gland area connected to NPLP were newly discovered as shown in FIG. 16. Ten weeks-old male Prox1-GFP mouse was subjected to tissue clearing. The head was sampled, fixed, and decalcified following the method for FIG. 12. After decalcification, the sample were incubated in CUBIC-L (T3740, Tokyo Chemical Industry Co. Ltd) solution for a week at 37 Celsius degree for tissue clearing. After tissue clearing step, the sample was washed with PBS for a day. To label lymphatic vessels, the sample was immersed in 5% normal donkey serum in which LYVE1 antibody (11-034, Angiobio) was dissolved at 1:200 ratio for 7 days at room temperature with gentle shaking. After primary antibody incubation and PBS washing, the sample was incubated to normal donkey serum containing Alexa-594 conjugated anti rabbit IgG antibody (Jackson ImmunoResearch) was dissolved at 1:100 for 4 days in room temperature with gentle shaking. After secondary antibody incubation and following washing with PBS, refraction index was matched by submerging the sample in D-PROTOSS solution (Ku et al., Nature methods, 2020) for 48 hours. The cleared sample was imaged with a light-sheet microscope (LightSheet7, Zeiss) with EC Plan Neofluar 5×/0.16 lens. Around the pituitary gland area, the blunt end of LV was observed (FIG. 16). These LVs run along the cavernous sinus and were connected to NPLP (FIG. 16B). 3D video of this connecting LV is shown in FIG. 17 (green arrowheads)

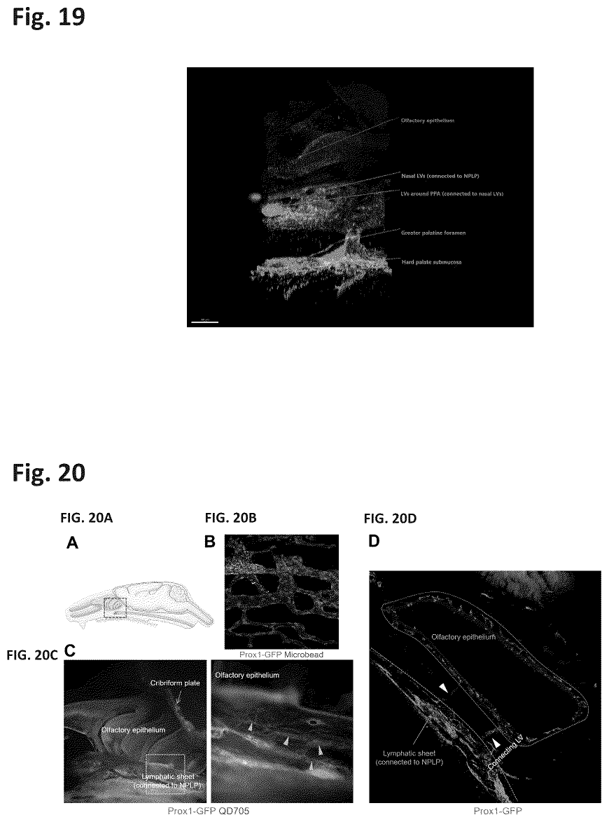

Example 8: Connection between LVs around the pterygopalatine artery (PPA) and posterior nasal lymphatic plexus which is connected to NPLP are newly discovered. (FIG. 18). The procedures for Example 7 were applied to 10 weeks-old male Prox1-GFP mice. Here, the Prox1+/LYVE1+LV is located along the pterygopalatine artery (PPA), has a blunt end at the basal meninges, and is connected to the posterior nasal lymphatic plexus. The lymphatic valve (white arrowhead, high Prox1⁺ signal), was located at the connection point (FIG. 18). In addition, some of these LVs run through the greater palatine foramen, and connect to the lymphatic plexus in the submucosa of the hard palate (FIG. 18 and FIG. 19).

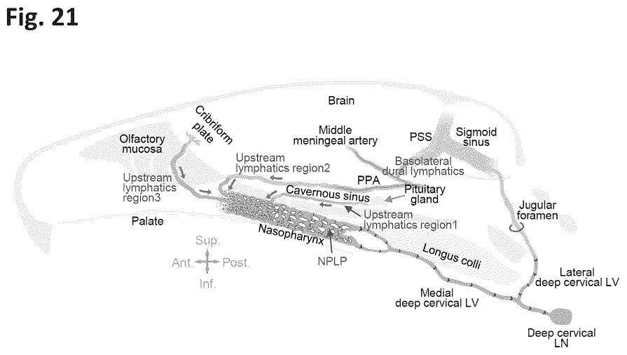

Example 9: The Prox1+ LVs located below the olfactory epithelium are connected to the posterior nasal lymphatic plexus, which is connected to the NPLP (FIG. 20, reddish-yellow and white arrowheads). These findings indicate that CSF can be drained through the LVs in the olfactory mucosa and the posterior nasal lymphatic plexus. A summary diagram of three new lymphatic routes from the intracranial cavity to extracranial NPLP is shown in FIG. 21. These are (1) the LVs initiated from the pituitary gland area connected to the NPLP, (2) the LVs run along the pterygopalatine artery (PPA) connecting to the NPLP, and (3) the LVs located anterior skull base run along the foramen of cribriform plate connecting the lymphatic vessels in the olfactory mucosa which is connected to posterior nasal lymphatic plexus and the NPLP. Thus, NPLP serves as a hub for CSF drainage.

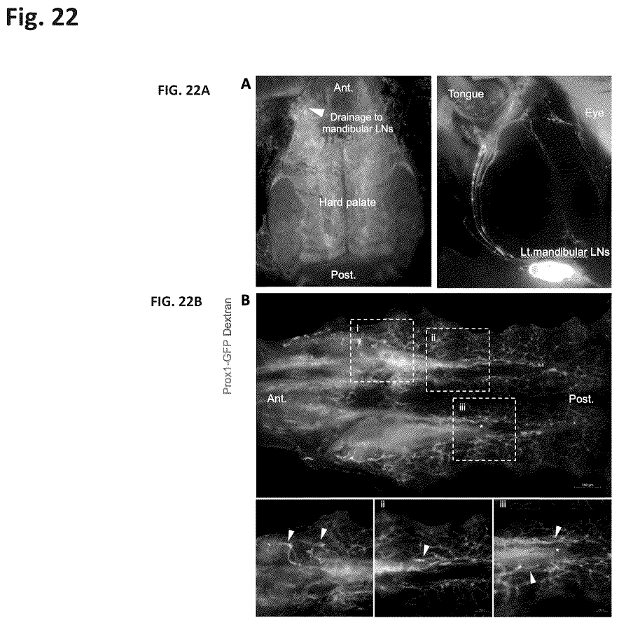

Example 10: The same procedure in Example 1 was applied to an anesthetized 10 weeks-old male Prox-1 GFP mouse, and then the mouse was sacrificed by cutting the abdominal aorta at 60 min after the injection. LVs and the dextran in the hard palate submucosa and mandibular LN were imaged using a fluorescence stereo zoom microscope (FIG. 22). We found that LVs in hard palate submucosa were connected to mandibular LN. The injected dextran was detected not only in the mandibular LN but also within lymphatic vessels in the submucosa of hard palate (FIG. 22A and FIG. 22B).

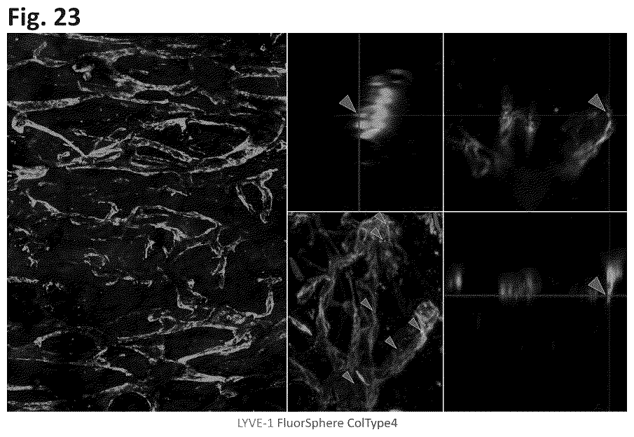

Primate (Cynomolgus monkey) was subjected to infuse fluorescent microspheres (ThermoFisher, F8801) through cisterna magna (2 mL, 25 L/min) under isoflurane anesthesia. 6 hours after infusion, blood was perfused with PBS and 2% PFA, then the hard palate was collected. We found the CSF-derived fluorescent microspheres were located within the lymphatic vessels of hard palate (FIG. 23). These findings indicate that the LVs in the hard palate serve as a drainage route of CSF, which is connected to the mandibular LN. In addition, these LVs can be subjected to modulate to facilitate CSF drainage.

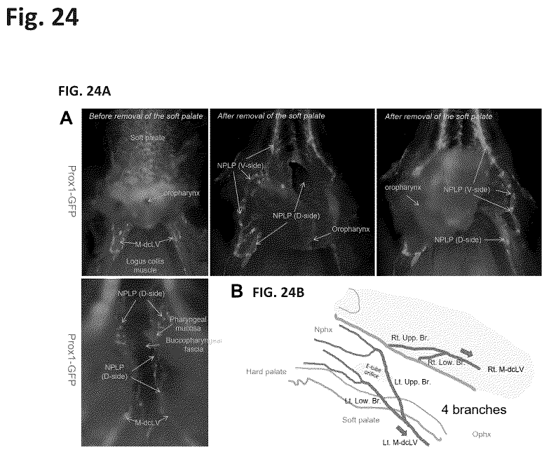

Example 10: IFS analysis reveals 4 lymphatic branches connecting NPLP and dcLVs in the Prox1-GFP mouse (FIG. 24). Removal of the soft palate clearly shows 4 lymphatic branches connecting NPLP and dcLVs (FIG. 24), implying that CSF outflow to dcLVs via NPLP.

Example 11: In collaboration with the National Primates center in Korea Research Institute of Bioscience and Biotechnology, 9 heads of Cynomolgus monkeys were obtained in 2022. The samples were perfused with ice-cold saline. The decapitated head samples emerged with 4% paraformaldehyde (PFA) for 2 hours at 4 Celsius degree. Then, the samples were emerged with 2% PFA for 12 hours at 4 Celsius degree. Then, the retropharyngeal LN which is equivalent to dcLN in the mice were harvested. After removing the lymph nodes, the heads emerged with 0.5M EDTA, pH8.0 (Welgene) for 3 weeks at 4 Celsius degree. Every 4 days, EDTA was changed with a new EDTA solution. After decalcification, the heads were trimmed. The anterior boundary was the choana, while the posterior boundary was the occipital bone. The dorsal boundary was the optic nerve, while the ventral boundary was the uvula. Then trimmed heads were sagittally cut in half. Brain was removed from the skull. The sample submerged in 30% of sucrose solution for 72 hours.

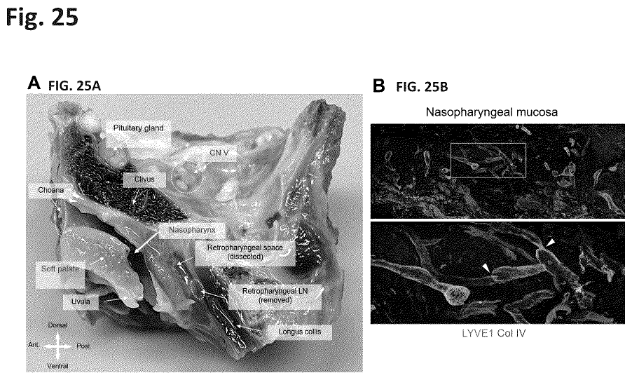

The nasopharynx was located just below the clivus which is one of the elements of skull base (FIG. 25). Ventral part of the nasopharyngeal mucosa is well distinguished from the soft palate (FIG. 25). The retropharyngeal lymph node was supposed to be located at the white dotted-circle in FIG. 25.

To get an IFS image, the fixed and dehydrated nasopharynx was coronally cut using a blade, and this sample was embedded and frozen in a frozen section medium (Leica) and cut into 30 μm section using Cryocut Microtome (Leica). To get whole mount tissue image, nasopharyngeal mucosa was carefully separated from skull base and soft palate. After PBS washing, the sample was incubated in 5% normal donkey serum (017-000-121, Jackson ImmunoResearch) for 1 hour at room temperature. To label lymphatic vessel markers, the nasopharyngeal mucosa was immersed in 5% normal donkey serum in which LYVE-1 antibody (DP3500, OriGene) and Collagen type IV antibody (AB769, Sigma) were dissolved at 1:400 at 4 Celsius degrees for 12 hours. After PBS washing, the tissue block was incubated to normal donkey serum containing Alexa-594 conjugated anti-goat IgG antibody and Alexa-647 conjugated anti-rabbit IgG antibody (Jackson ImmunoResearch) for 12 hours in 4 Celsius degree. After PBS washing, the tissue block was incubated with mounting medium (H1200, Vector). Image was acquired by confocal microscope (LSM 800, Zeiss) with Plan-Apochromat 10×/NA 0.45 lens.

Similar to the mice, NPLP is present in the submucosa of nasopharynx of Cynomolgus monkey (FIG. 25).

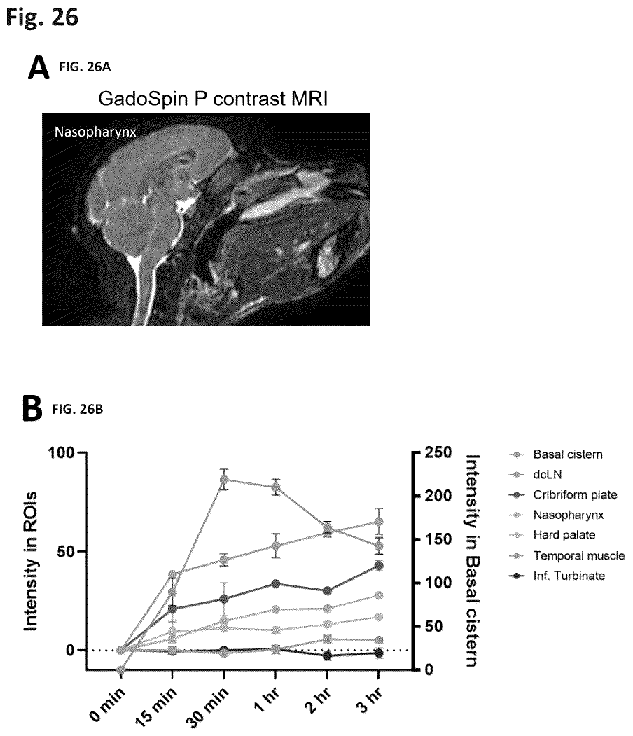

Furthermore, contrast-enhanced MR images were acquired using Cynomolgus monkey after MR contrast enhancement agent, Gadospin P, infusion through the cisterna magna. To infuse Gadospin P, infusion needle was positioned with computed tomography (CT). After needle position was confirmed, Gadospin P (10% dissolved in saline, 25 μL/min) was infused. Images with FLAIR T2 MRI (3T, Phillips Medical System) were acquired for 3 hours 30 mins before and after Gadospin P infusion. The primates were under isoflurane anesthesia and physiological monitoring was conducted by a veterinarian. We found signal enhancement within the nasopharynx in the primate (FIG. 26).

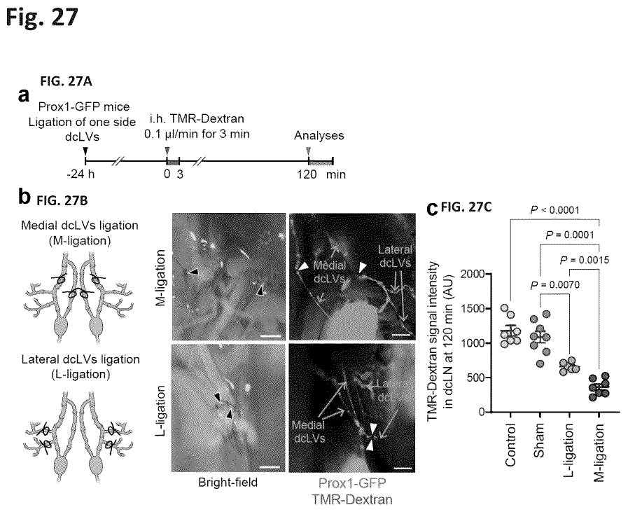

Example 12: Functional studies: To estimate which side dcLV drains more CSF, a ligation of one side of dcLV was performed in the 10 weeks-old Prox1-GFP mice of both sexes. After dissecting the sternocleidomastoideole, both sides of dcLVs carefully, one-side of dcLV was ligated with a 10-0 polypropylene suture (W2794, Ethicon) while the other-side of dcLV was sham-ligated. One day after ligation, TMR-dextran was injected into cisterna magna following the methods of Example 1. Here, compared with the dextran signal of sham operation, the M-dcLV ligation markedly reduced the dextran signal in the dcLN, while the L-dcLV ligation mildly reduced the Dextran signal in the dcLN (FIG. 27). These results imply that the CSF drainage through the NPLP/M-dcLV route is more than through the basolateral mLV/L-dcLV route, highlighting the importance of NPLP. P values were calculated by one-way ANOVA test followed by Turkey's post-hoc test.

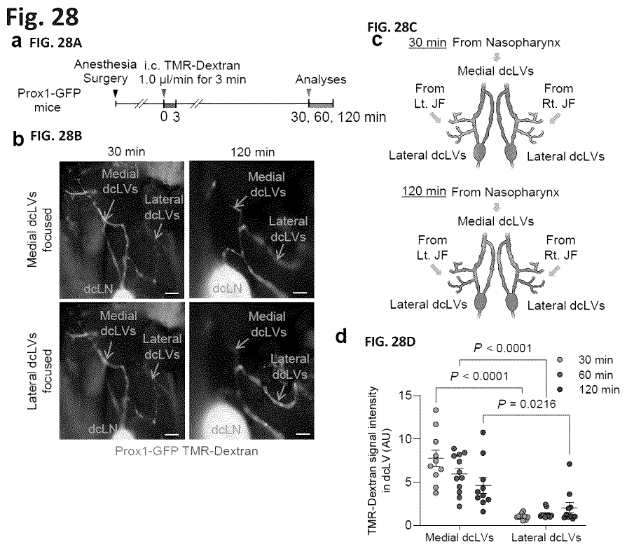

The same injection into the intracranial cavity was applied to the 10 weeks-old Prox1-GFP mice of both sexes for measuring the speed of CSF drainage into the dcLV (FIG. 27). The Dextran signal was measured in the dcLV. Compared to the Dextran signal in the L-dcLV, the Dextran signal were 7.9-fold, 5.2-fold and 2.1-fold higher at 30 min, 60 min, and 120 min after the injection (n=10-12 per each group) (FIG. 28). These results imply that the CSF drainage through the NPLP/M-dcLV route is faster than through the basolateral mLV/L-dcLV route, emphasizing the importance of NPLP. P values were calculated by one-way ANOVA test followed by Turkey's post-hoc test.

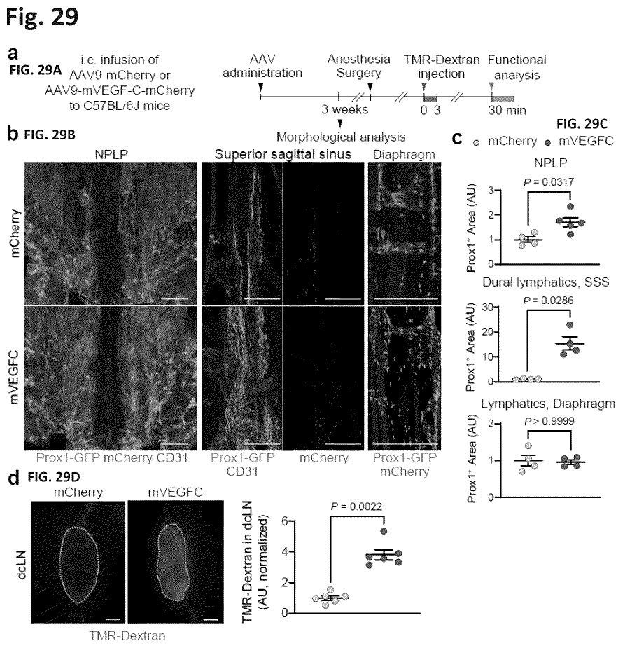

Example 13: To test whether NPLP can be modified by an exogeneous agent, 1.1×10¹³ GC/mL of AAV-mVEGF-C-mCherry was injected (1.0 μl/min for 3 min) into the intracranial cavity via cisterna magna of 10 weeks-old Prox1-GFP female mice. As a control, the same amount of AAV-empty vector was injected. Three weeks after the injection, the NPLP of both groups compared. Compared to the AAV-empty group, the AAV-mVEGF-C group exhibited increased Prox1+ area. (FIG. 29). These findings imply that NPLP can be modified by an exogeneous agent. The amount of drained CSF in dcLN is enhanced in AAV-VEGF-C group (FIG. 29). This finding implies that modification of CSF lymphatic drainage route including NPLP can enhance CSF drainage.

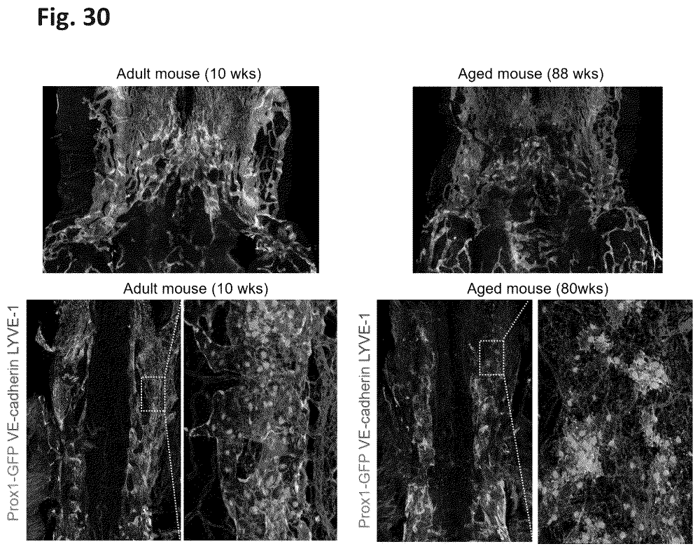

Example 14: To examine whether aging alters NPLP, we performed IFS on the NPLP in adult and aged mice. Compared to adult (10 weeks-old male) mice, aged (80-88 weeks-old) mice exhibited 1) reduction of lymphatic valves in the NPLP, 2) regression of Prox1⁺ lymphatic vessels, 3) increase of LYVE1, 4) fragmentation of dorsal NPLP at the skull base portion (FIG. 30).

Furthermore, we analyzed transcriptome of lymphatic endothelial cells (LECs) in the young and aged NPLP. After anesthesia, mice were perfused with ice-cold PBS, and the nasopharyngeal submucosa was isolated and pooled in DMEM/F12 medium (Gibco). The nasopharyngeal submucosa was cut into small pieces and incubated in dissociation buffer containing 1 mg/ml of collagenase IV (Roche), 1 mg/ml of dispase (Gibco), and 0.1 mg/ml DNase I (Gibco) at 37° C. for 30 min with gentle inverting every 10 min. Digested samples were filtered through a 70 μm strainer and 2% FBS was added to stop digestion. The cells were centrifuged for 8 min with 500× g and resuspended with PBS for washing. To exclude dead cells, 1:1000 of Ghost dye (TONBO bioscience) was added to resuspended cells for 15 min at 4° C. Then, PBS were added for washing followed by staining with phycoerythrin/Cy7 anti-mouse CD326 (Ep-CAM, 118216, Biolegend) antibody, APC anti-mouse podoplanin antibody (127410, Biolegend), and phycoerythrin-labeled anti-mouse CD31 antibody (102508, Biolegned). CD31+PDPN+ cells were considered as LECs and sorted by a FACS Aria Fusion (Beckton Dickinson). Sorted LECs were directly placed to each well in a 96-well plate containing a lysis buffer. Plates were snap froze with liquid nitrogen and stored at −80° C. Following Smart-Seq3 protocol plate-based single-cell libraries were generated. Shortly, mRNAs from lysed cells were reverse transcribed. cDNAs were amplified and purified with Ampure XP beads (Beckman Coulter). Purified cDNAs were diluted (100 pg/μl) and tagmented using a Tn5 tagmentation mix (Illumina). Using custom index primers, tagmented products were amplified and then pooled into a single tube. After final cleanup with Ampure XP beads, libraries were analyzed by tape station for quality control. Libraries passing the quality control were sequenced with Illumina High-X platform. Sequenced libraries were demultiplexed and aligned to mouse reference genome (mm10) by STAR (version 2.7.9.a). Then, the featureCount (version v2.0.1) function from Subread package was used to merge the aligned files and to build raw read count matrices. For cell quality control, cells detected with less than 2,000 genes and cells with more than 10% of total reads mapped to mitochondrial genes were considered low quality/dead cells and discarded. At the gene level, genes expressed in less than 3 cells were removed from the expression matrix. For clustering and visualization of single cells, R package Seurat was used (version 4.1.0). Briefly, log 2 normalizations were applied after dividing each count for a gene in a cell by total number of counts in a given cell, with multiplication of 1×10⁴ and addition of pseudo-count of 1. Consequently, resulting expression matrixes were transformed to have values similar to log transformed counts per million. Then, the top 2,000 genes with the highest variability in each dataset were selected by the FindVariableFeatures function with options: selection.method=“vst”. Those highly variable genes were scaled and centered while regressing out confounding variables such as number of total counts and the percentage of reads mapped to mitochondrial genes. In addition, module scores for dissociation induced genes and ribosomal genes were calculated by AddModuleScore function and regressed.

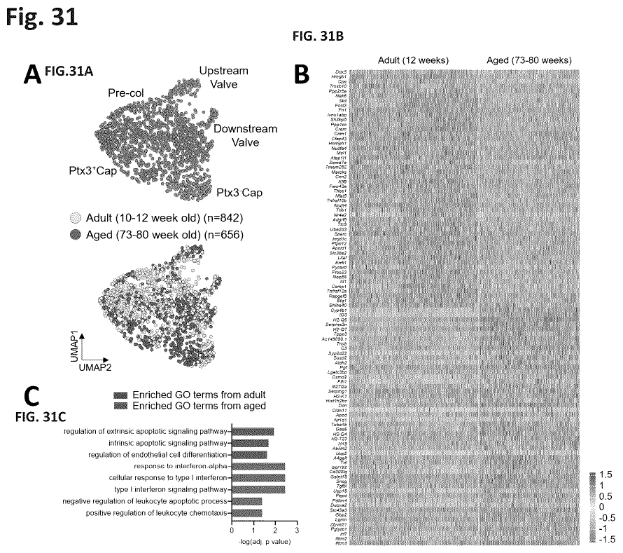

For visualization in 2-dimensional space, principal component analysis was performed, and the top 15 principal components were used as input for Uniform Manifold Approximation and Projection (UMAP). For neighborhood identification and cluster assignment, shared nearest neighborhood graph was built by using the top 15 principal components and Louvain algorithm was applied. For identifying differentially expressed genes between cells, we used the FindMarkers function in Seurat with options: test.use=“MAST”, logfc.threshold=0.3, min.pct=0.3. While performing differential expression testing, we excluded dissociation induced genes, mitochondrial and ribosomal genes. In merging adult and aged mouse datasets, no batch correction method was used, as no evident batch effect was observed for clustering. We found the 5 LECs clusters were preserved but genes involved in apoptosis and inflammation were differently expressed which can be interpreted the aged NPLP was more pro-apoptotic and pro-inflammatory (FIG. 31).

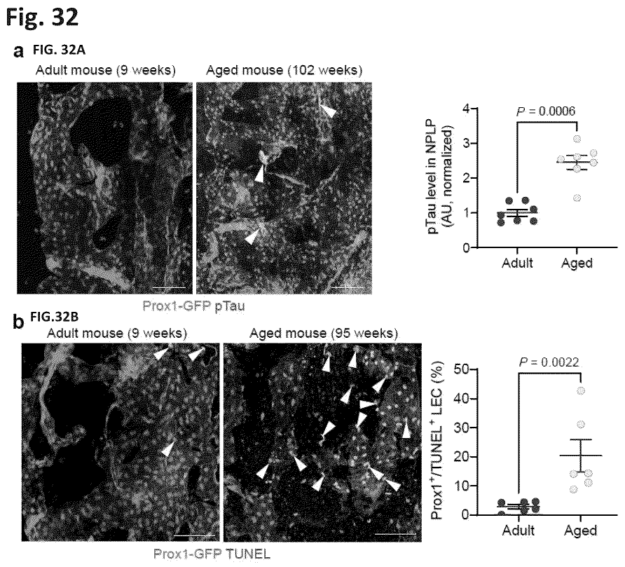

We also performed IFS in the aged NPLP as same procedure described in Example 6 to measure phosphorylated tau (ptau) and apoptotic lymphatic endothelial cells. The ptau is detected by using anti-phospho-tau (mouse monoclonal (AT8) antibody, MN1020, Thermo) and is increased in the aged NPLP (FIG. 32). The number of apoptotic lymphatic endothelial cells is detected using TUNEL kit (12156792910, Merck) and is increased with aging (FIG. 32). These results indicate the increased apoptosis and accompanying changes (upregulation of type I interferon signaling) may be caused by increased CSF ptau.

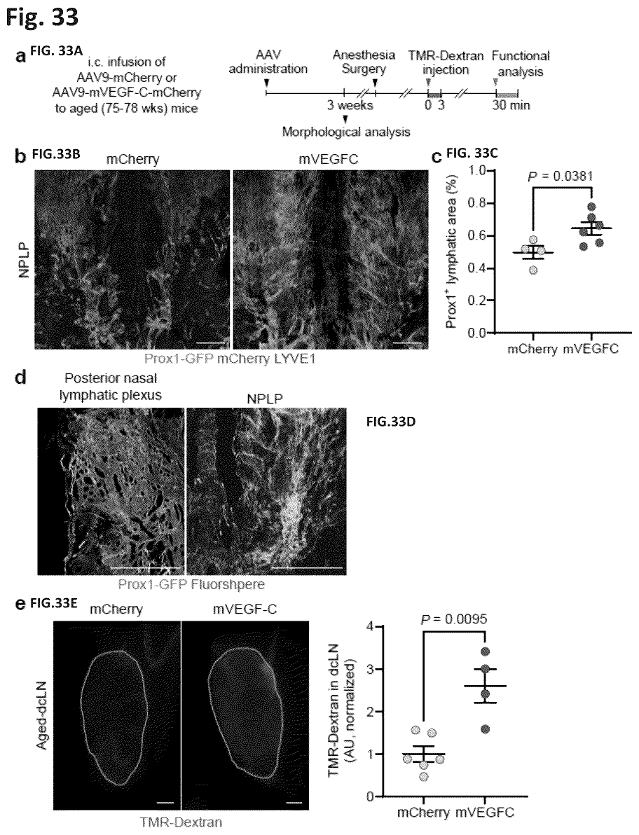

Example 15: To test whether the aged NPLP can be modified by an exogeneous agent, 1.1×10¹³ GC/mL of AAV-VEGF-C-mCherry was injected (1.0 μl/min for 3 min) into the intracranial cavity via cisterna magna of 75-78 weeks-old Prox1-GFP female mice. As a control, the same amount of AAV-empty vector was injected. Three weeks after the injection, the NPLP of both groups were compared. Compared to the AAV-empty group, the AAV-VEGF-C-mCherry group exhibited 1) increased Prox1⁺ area and 2) enhanced CSF drainage in dcLN (FIG. 33). Furthermore, CSF tracers are observed in lymphatic vessels after AAV-mVEGF-C infection. These findings imply that impaired NPLP with aging can be modified by an exogeneous agent.

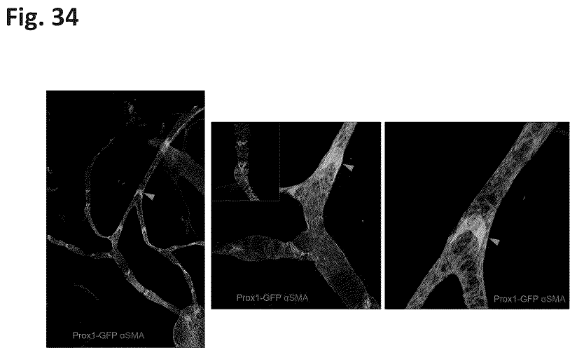

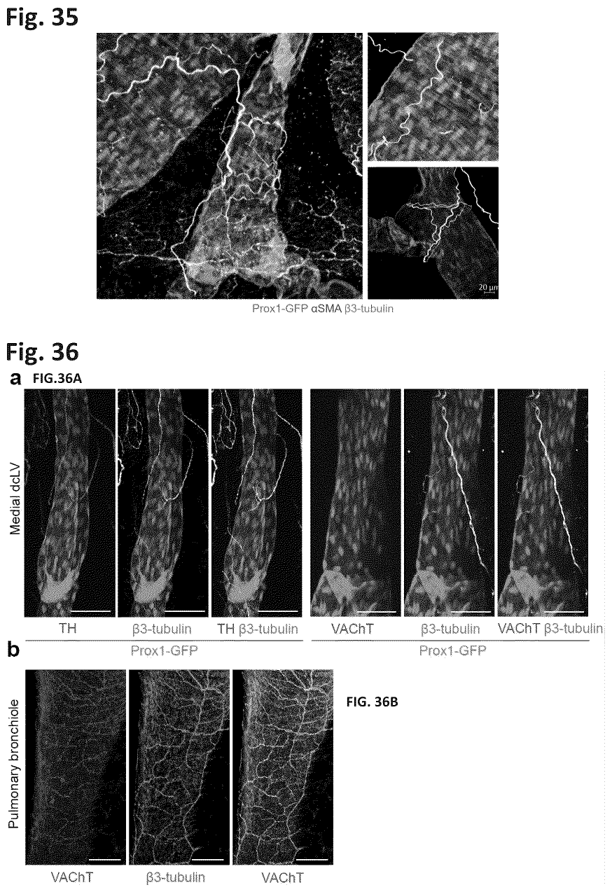

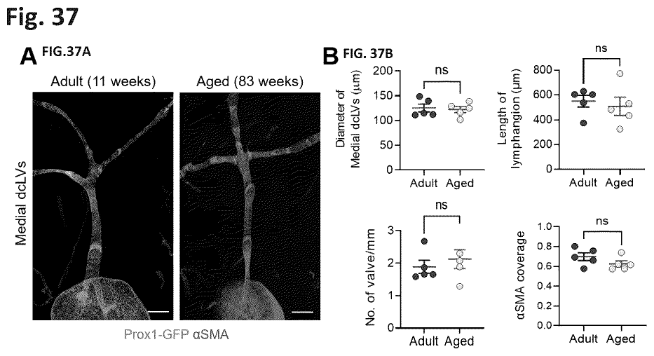

Example 16: IFS analysis reveals that dcLVs have well-developed, semi-luminal shaped lymphatic valves and lymphangions, and are finely covered with circular smooth muscle cells (FIG. 34). Peripheral nerve fibers are distributed along the dcLVs (FIG. 35). The innervating peripheral nerve fibers are sympathetic nerve fibers but not parasympathetic nerve fibers (FIG. 36). There is no prominent change of structure of surrounding smooth muscle cells and number of lymphatic valves in aged Prox1-GFP mice (FIG. 37).

The dcLVs are strongly but transiently contracted by 0.1 and 1.0 M of potassium chloride (KCl) (FIG. 38). In addition, G protein-coupled receptor agonist phenylephrine or nitric oxide donor (sodium nitroprusside) contracts or dilates dcLVs and the amount of CSF tracer can be modulated (FIG. 39). Furthermore, we found that low dosage of phenylephrine (10 nM) increased the amount of drained CSF but reduced the amount of drained CSF with high dosage (5 mM) measured by accumulated CSF tracer in dcLN (FIG. 40). Low dosage of nitric oxide donor (3 μM), sodium nitroprusside, enhanced CSF drainage but no change is observed with 30 μM of nitric oxide donor (FIG. 40).

These findings imply that contraction and relaxation of the circular smooth muscles covering the dcLVs can be regulated by 1) stimulation and inhibition of the smooth muscle cells or peripheral nerves, 2) neurotransmitters, 3) mechanical stimulators, and 4) gentle massage. Basically, CSF drainage can be facilitated by extracranial manipulations, administration of agents, and regulators (FIG. 41). Considering that there is no prominent change in aged dcLVs, the reduced CSF drainage can be enhanced by extracranial manipulations of dcLVs.

Together, these examples provide information on where CSF drainage occurs and how CSF drainage can be facilitated by extracranial approaches and methods. Because this invention discloses that 1) NPLP is a newly discovered hub for CSF drainage through the skull base and cribriform plate, 2) the hard palate submucosa is a newly discovered route for CSF drainage to the mandibular LNs, 3) impaired CSF drainage with aging can be enhanced by rescuing CSF drainage route by exogenous agent such as VEGF-C, and 4) enhanced contraction and relaxation of the circular smooth muscles covering the dcLVs can be regulated by stimulation and inhibition of the ensheathing smooth muscle cells, peripheral nerves, neurotransmitters, mechanical stimulators, as CSF drainage can be facilitated by extracranial manipulations, administration of agents, and related regulators for preventing and treating neurodegenerative diseases including Alzheimer's disease (FIG. 42).

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

### CITED REFERENCES

1. Ransohoff, R. M., & Engelhardt, B. (2012). The anatomical and cellular basis of immune surveillance in the central nervous system. Nat Rev Immunol, 12(9), 623-635. https://doi.org/10.1038/nri3265
2. Proulx, S. T. (2021). Cerebrospinal fluid outflow: a review of the historical and contemporary evidence for arachnoid villi, perineural routes, and dural lymphatics. Cell Mol Life Sci, 78(6), 2429-2457. https://doi.org/10.1007/s00018-020-03706-5
3. Bettcher, B. M., Tansey, M. G., Dorothee, G., & Heneka, M. T. (2021). Peripheral and central immune system crosstalk in Alzheimer disease—a research prospectus. Nat Rev Neurol, 17(11), 689-701. https://doi.org/10.1038/s41582-021-00549-x
4. Aspelund, A., Antila, S., Proulx, S. T., Karlsen, T. V., Karaman, S., Detmar, M., Wiig, H., & Alitalo, K. (2015). A dural lymphatic vascular system that drains brain interstitial fluid and macromolecules. J Exp Med, 212(7), 991-999. https://doi.org/10.1084/jem.20142290
5. Louveau, A., Smirnov, I., Keyes, T. J., Eccles, J. D., Rouhani, S. J., Peske, J. D., Derecki, N. C., Castle, D., Mandell, J. W., Lee, K. S., Harris, T. H., & Kipnis, J. (2015). Structural and functional features of central nervous system lymphatic vessels. Nature, 523(7560), 337-341. https://doi.org/10.1038/nature14432
6. Ahn, J. H., Cho, H., Kim, J. H., Kim, S. H., Ham, J. S., Park, I., Suh, S. H., Hong, S. P., Song, J. H., Hong, Y. K., Jeong, Y., Park, S. H., & Koh, G. Y. (2019). Meningeal lymphatic vessels at the skull base drain cerebrospinal fluid. Nature, 572(7767), 62-66. https://doi.org/10.1038/s41586-019-1419-5
7. Pan, W. R., Suami, H., Corlett, R. J., & Ashton, M. W. (2009). Lymphatic drainage of the nasal fossae and nasopharynx: preliminary anatomical and radiological study with clinical implications. Head Neck, 31(1), 52-57. https://doi.org/10.1002/hed.20926
8. Ma, Q., Ineichen, B. V., Detmar, M., & Proulx, S. T. (2017). Outflow of cerebrospinal fluid is predominantly through lymphatic vessels and is reduced in aged mice. Nat Commun, 8(1), 1434. https://doi.org/10.1038/s41467-017-01484-6
9. Jacob, L., de Brito Neto, J., Lenck, S., Corcy, C., Benbelkacem, F., Geraldo, L. H., Xu, Y., Thomas, J. M., El Kamouh, M. R., Spajer, M., Potier, M. C., Haik, S., Kalamarides, M., Stankoff, B., Lehericy, S., Eichmann, A., & Thomas, J. L. (2022). Conserved meningeal lymphatic drainage circuits in mice and humans. J Exp Med, 219(8). https://doi.org/10.1084/jem.20220035
10. Kipnis, J., Louveau, A., Da Mesquita, S. (2019). US 2019-0269758 A1, University of Virginia Patent Foundation (UN162191): U.S. Patent and Trademark Office
11. Kipnis, J. Da Mesquita, S. (2021), US 2021-0311076 A1, University of Virginia Patent Foundation (UN162191): U.S. Patent and Trademark Office

1. A method of increasing outflow of cerebral spinal fluid (CSF) from central nervous system comprising repairing or enlarging nasopharyngeal lymphatic plexus (NPLP) comprising:
determining a subject in need of increased CSF outflow; and
administering an effective amount of NPLP flow agent to the subject in need, whereby the amount of the agent repairs or enlarges NPLP of the subject, thereby increasing CSF outflow from the central nervous system to systemic circulation in the subject.

2. The method of claim 1, wherein determining the subject in need of increased CSF outflow comprises determining the subject to have a neurodegenerative disease or condition, determining the subject to have a risk factor for the neurodegenerative disease or condition, or both.

3. The method of claim 1, wherein the disease or condition is cognitive decline with aging, Alzheimer's disease, Parkinson's disease, Huntington's disease, or stroke.

4. The method of claim 1, wherein the agent is VEGFR3 agonist.

5. The method of claim 4, wherein the agent is VEGF-C or VEGF-D, an analog, variant, or fragment thereof, or a combination of any of these.

6. The method of claim 1, wherein the agent is fibroblast growth factor 2 (FGF-2), insulin-like growth factor 1 (IGF-1), hepatocyte growth factor (HGF), endothelin-1 (ET-1), angiopoietin-1, Tie2 agonist, neuropilins, or prostaglandin E2.

7. The method of claim 1, wherein the agent is a protein, or a genetic vector that carries a gene encoding an agent polypeptide.

8. The method of claim 7, wherein the gene encodes vascular growth factor-C, angiopoietin-1, or Tie2 agonist.

9. The method of claim 1, wherein the agent is administered selectively at or near NPLP-dcLV space.

10. The method of claim 1, wherein the NPLP-dcLV space is located in nasopharyngeal mucosa.

11. The method of claim 1, wherein the agent is administered selectively at or near hard palate submucosa-mandibular lymph node space.

12. The method of claim 11, wherein the hard palate submucosa-mandibular lymph node space is located in hard palate mucosa.

13. The method of claim 1, wherein the agent is administered to the subject intrathecally to CSF space or trans-nasally to nasopharynx or trans-orally to hard palate submucosa.

14. A method of increasing outflow of cerebral spinal fluid (CSF) from central nervous system comprising increasing contracting-relaxing of dcLV comprising:
determining a subject in need of increased CSF outflow; and
administering an effective amount of dcLV flow agent to the subject, whereby the amount increases contracting-relaxing of dcLV of the subject, thereby increasing CSF flow from the central nervous system to systemic circulation in the subject.

15. The method of claim 14, wherein determining the subject in need of increased CSF flow comprises determining the subject to have a neurodegenerative disease or condition, determining the subject to have a risk factor for the neurodegenerative disease or condition, or both.

16. The method of claim 14, wherein the disease or condition is cognitive decline with aging, Alzheimer's disease, Parkinson's disease, Huntington's disease, or stroke.

17. The method of claim 14, wherein the agent is G protein-coupled receptor agonist phenylephrine or nitric oxide donor.

18. The method of claim 14, wherein the agent is administered to the subject transcervically, percutaneously, topically to neck muscles or neck nodes or neck spaces, or wherein the agent is mechanical application to the side of the neck.

19. A method of preventing or treating or ameliorating a neurodegenerative disease or condition in a subject comprising repairing or enlarging nasopharyngeal lymphatic plexus (NPLP) comprising:
determining the subject in need of increased CSF outflow from the central nervous system; and
administering an effective amount of NPLP flow agent to the subject in need, whereby the amount of the agent repairs or enlarges NPLP of the subject, thereby increasing CSF outflow from the central nervous system to systemic circulation in the subject.

20. The method of claim 19, wherein the disease or condition is cognitive decline with aging, Alzheimer's disease, Parkinson's disease, Huntington's disease, or stroke.

21.-40. (canceled)
